(19) United States
(12) Patent Application Publication
(10) Pub. No.: US 2023/0248824 A1
(43) Pub. Date: Aug. 10, 2023

(54) IMMUNE CELLS WITH INCREASED GLYCOLYTIC FLUX

(71) Applicant: The Board of Trutees of the Leland Stanford Junior University, Stanford, CA (US)

(72) Inventors: Dorota Danuta KLYSZ, Palo Alto, CA (US); Crystal L. MACKALL, Palo Alto, CA (US)

(21) Appl. No.: 18/004,638

(22) PCT Filed: Jul. 8, 2021
(86) PCT No.: PCT/US2021/040875

§ 371 Date: Jan. 6, 2023

Related U.S. Application Data
(60) Provisional application No. 63/049,946, filed on Jul. 9, 2020.

(51) Int. Cl. A61K 39/00; C07K 14/705; C07K 14/725; C12N 5/0783; A61P 35/00

(52) U.S. Cl. CPC A61K 39/4611; C07K 14/705; C07K 14/7051; C12N 5/0636; A61P 35/00; A61K 39/4631; A61K 39/4637; C12N 2510/00; A61K 2239/47; C07K 2319/33

(57) ABSTRACT

The present disclosure generally relates to, inter alia, recombinant immune cells that have been engineered to express elevated levels of one or more glucose transporters, and particularly relate to engineered immune cells exhibiting increased glycolytic flux and/or enhanced effector functions. Also provided are methods for generating a population of engineered immune cells with enhanced effector function, pharmaceutical compositions the same, as well as methods and kits for the prevention and/or treatment of a health condition in subjects in need thereof.

### CROSS-REFERENCE TO RELATED APPLICATION

The present application claims priority to U.S. Provisional Patent Application Ser. No. 63/049,946, filed on Jul. 9, 2020, the disclosure of which is incorporated by reference herein in its entirety, including any drawings.

### FIELD

The present disclosure generally relates to, inter alia, recombinant immune cells that have been engineered to express elevated levels of one or more glucose transporters, and particularly relate to engineered immune cells exhibiting increased glycolytic flux and/or enhanced effector functions. Also provided are methods for generating engineered immune cells with enhanced effector function, pharmaceutical compositions the same, as well as methods and kits for the prevention and/or treatment of a health condition in subjects in need thereof.

### BACKGROUND

Adoptive transfer of genetically modified immune cells has emerged as a potent therapy for various malignancies. For example, current modalities of adoptive T cell therapy include cells modified to express receptors specific for cancer antigens, such as chimeric antigen receptors (CARs) and high-affinity T cell receptors (TCRs). Upon exposure to the cancer antigen, the modified T cells exhibit cytolytic activity and/or send signals to initiate an immune response against the cancer.

In adoptive T cell therapies, modified T cells are generally activated by exposure to the cognate antigen in vitro or ex vivo, expanded, and then administered to the subject, where they proliferate and have anticancer activity. Recent clinical trials using CAR-modified T cells (CAR-T cells) specific for the CD19 molecule on B-cell malignancies demonstrated marked disease regression in a subset of patients with advanced cancers. However, extending this therapy to other types of cancers, especially to solid tumors poses several challenges. For example, overstimulation due to prolonged antigen recognition and exposure to inflammatory signals can cause the T cells to lose effector function, a phenomenon called “T cell exhaustion.” Additionally, the tumor microenvironment elicits a number of tolerance and immunosuppression mechanisms that can reduce the effectiveness of adoptive cell therapies.

Thus, new compositions and strategies are needed for generating improved therapeutic cells for adoptive cell therapy. The presently disclosed aspects and embodiments address these needs and provide other related advantages.

### SUMMARY

Provided herein, inter alia, are novel methods and compositions for the prevention and/or treatment of various health conditions. In particular, described herein are immune cells that have been engineered to express elevated levels of glucose transporters. In some embodiments, the engineered immune cells exhibit increased glycolytic flux and/or enhanced effector functions. Also provided are methods for generating a population of engineered immune cells with enhanced effector function, and pharmaceutical compositions containing such a population of engineered immune cells with enhanced effector function, as well as methods and kits for the prevention and/or treatment of a health condition in subjects in need thereof.

In one aspect, provided herein are methods for generating an engineered immune cell with enhanced effector function, the method comprising introducing into the immune cell a nucleic acid and/or a polypeptide capable of conferring increased expression of one or more glucose transporters in the immune cell.

Non-limiting exemplary embodiments of the disclosed methods can include one or more of the following features. In some embodiments, the nucleic acid includes a sequence encoding one or more glucose transporters. In some embodiments, the one or more glucose transporters is selected from the group consisting of GLUT1, GLUT2, GLUT3, GLUT4, GLUT6, GLUT8, GLUT9, GLUT10, GLUT11, GLUT12, and functional variants of any thereof. In some embodiments, the one or more glucose transporters is selected from the group consisting of GLUT1, GLUT2, GLUT3, GLUT4, and functional variants of any thereof. In some embodiments, the coding sequence of the one or more glucose transporters is operably linked to a transcription control element. In some embodiments, the nucleic acid is incorporated into an expression cassette or a vector. In some embodiments, the vector is a viral vector derived from a lentivirus, an adeno-virus, an adeno-associated virus, a baculovirus, or a retrovirus. In some embodiments, the nucleic acid is incorporated into a nucleic construct for use in CRISPR-mediated knock-in procedure. In some embodiments, the immune cell is T lymphocyte, a natural killer (NK) cell, or a natural killer T cell (NKT). In some embodiments, the T lymphocyte is a CD8+ T cytotoxic lymphocyte cell selected from the group consisting of naive CD8+ T cells, central memory CD8+ T cells, effector memory CD8+ T cells, effector CD8+ T cells, CD8+ stem memory T cells, bulk CD8+ T cells. In some embodiments, the lymphocyte is a CD4+ T helper lymphocyte cell selected from the group consisting of naïve CD4+ T cells, central memory CD4+ T cells, effector memory CD4+ T cells, effector CD4+ T cells, CD4+ stem memory T cells, and bulk CD4+ T cells.

In some embodiments, the enhanced effector function is selected from the group consisting of growth rate (proliferation), death rate, death rate type, target cell inhibition (cytotoxicity), target cell killing, target cell survival, cluster of differentiation change, macrophage activation, B cell activation, cytokine production, in vivo persistence, and increased glycolytic flux. In some embodiments, the enhanced effector function comprises increased production of interferon gamma (INFγ), tumor-necrosis factor α (TNFα), and/or interleukin-2 (IL-2).

In some embodiments, the increased expression of one or more glucose transporters in the immune cell confers increased mitochondrial activity and/or increased mitochondrial fitness of the immune cell.

In some embodiments, the methods of the disclosure further include introducing into the immune cell one or more recombinant immune receptors, such as a chimeric antigen receptor (CAR) or a T cell receptor (TCR).

In one aspect, provided herein are engineered immune cells produced by a method of the disclosure. Non-limiting exemplary embodiments of the cells described herein can include one or more of the following features. In some embodiments, the immune cell is in vitro, ex vivo, or in vivo. In some embodiments, the immune cell is an exhausted immune cell or a non-exhausted immune cell. In a related aspect, provided herein cell cultures comprising at least one engineered cell of the disclosure, and a culture medium.

In another aspect, provided herein are pharmaceutical compositions comprising a pharmaceutically acceptable excipient and a) an engineered cell of the disclosure; and/or b) a nucleic acid comprising a sequence encoding one or more glucose transporters selected from the group consisting of GLUT1, GLUT2, GLUT3, GLUT4, GLUT6, GLUT8, GLUT9, GLUT10, GLUT11, GLUT12, and functional variants of any thereof. In some embodiments, the composition includes an engineered cell of the disclosure, and a pharmaceutically acceptable excipient. Non-limiting exemplary embodiments of the pharmaceutical compositions described herein can include one or more of the following features. In some embodiments, the composition includes a nucleic acid encoding one or more glucose transporters, and a pharmaceutically acceptable excipient. In some embodiments, the composition includes the nucleic acid is encapsulated in a viral capsid, a liposome, or a lipid nanoparticle (LNP).

In another aspect, provided herein are methods for treating a health condition in a subject in need thereof, the method comprising administering to the subject a composition comprising: (a) an engineered immune cell of the disclosure; b) a nucleic acid comprising a sequence encoding one or more glucose transporters selected from the group consisting of GLUT1, GLUT2, GLUT3, GLUT4, GLUT6, GLUT8, GLUT9, GLUT10, GLUT11, GLUT12, and functional variants of any thereof and/or c) a pharmaceutical composition of the disclosure.

Non-limiting exemplary embodiments of the treatment methods described herein can include one or more of the following features. In some embodiments, the health condition is a proliferative disease, an autoimmune disorder, or an infection. In some embodiments, the subject is a mammalian subject. In some embodiments, the subject has or is suspected of having a proliferative disease, an autoimmune disease, or an infection.

In some embodiments, the proliferative disease is a cancer. In some embodiments, the administered composition confers enhanced effector function selected from the group consisting of growth rate (proliferation), death rate, death rate type, target cell inhibition (cytotoxicity), target cell killing, target cell survival, cluster of differentiation change, macrophage activation, B cell activation, cytokine production, in vivo persistence, and increased glycolytic flux. In some embodiments, the enhanced effector function includes increased production of interferon gamma (INFγ), tumor-necrosis factor a (TNFα), and/or interleukin-2 (IL-2). In some embodiments, the composition is administered to the subject individually (monotherapy) or in combination with a second therapy, wherein the second therapy is selected from the group consisting of chemotherapy, radiotherapy, immunotherapy, hormonal therapy, toxin therapy, or surgery.

In yet another aspect, provided herein are kits for the prevention and/or treatment of a condition in a subject in need thereof, the kit comprising: (a) an engineered immune cell of the disclosure; (b) a nucleic acid comprising a sequence encoding one or more glucose transporters selected from the group consisting of GLUT1, GLUT2, GLUT3, GLUT4, GLUT6, GLUT8, GLUT9, GLUT10, GLUT11, GLUT12, and functional variants of any thereof; and/or c) a pharmaceutical composition of the disclosure.

The foregoing summary is illustrative only and is not intended to be in any way limiting. In addition to the illustrative embodiments and features described herein, further aspects, embodiments, objects and features of the disclosure will become fully apparent from the drawings and the detailed description and the claims.

### BRIEF DESCRIPTION OF THE DRAWINGS

FIGS. 1A-1C graphically summarize the results of experiments performed to demonstrate that overexpression of GLUT1 increases cytokine secretion of exhausted and non-exhausted CAR T cells activated through their TCR or their CAR. FIG. 1A graphically summarize the results of experiments performed to illustrate increased IL-2 secretion by non-transduced (left panel) or HA-GD2 CAR T cells ⁺/⁻GLUT1 (right panel) assessed after 24-hour stimulation with anti-CD3/anti-CD28 beads in ratio 1:1 or 1:3 at Day 10 post-activation.

FIG. 1B graphically summarize the results of experiments performed to illustrate increased IFNγ Day 13 (left panel) and 14 (middle panel) and increased IL-2 (right) at Day 15 production after 24 hours of co-culture by control or GLUT1 overexpressing HA-GD2 CAR T cells in response to antigen-positive tumor cells evaluated by ELISA.

FIG. 1C graphically summarize the results of experiments performed to illustrate increased IFNγ (left) and IL-2 (right) secretion after stimulation with Nalm6 cell line by CD1928 CAR T cells ⁺/⁻ GLUT1 at Day 15 post-activation. Data are mean±s.e.m. of triplicate wells. P values determined by unpaired two-tailed t-test.

FIGS. 2A-2C graphically summarize the results of experiments performed to illustrate that GLUT1 overexpression increases proliferation and expansion of exhausted CAR T cells. FIG. 2A graphically summarize the results of experiments performed to illustrate increased proliferation and expansion of exhausted CAR T cells. On Day 14 post-activation HA-GD2 and HA-GD2 overexpressing GLUT1 CAR T cells were loaded with proliferation dye and 0.05×106 viable cells were cultured for 4 days in complete RPMI with IL-2. CAR T cell proliferation was monitored as a ratio of losing the proliferation dye.

FIG. 2B is a graph illustrating fold increase in total number as shown in FIG. 2A.

FIG. 2C is a graph illustrating difference in proliferation between tonic signaling HA-GD2 CAR+GLUT1+ and CAR−GLUT1+ T cells at Day 18. Data are mean±s.e.m. of duplicate wells. Representative histograms are shown.

FIGS. 3A-3F graphically summarize the results of experiments performed to illustrate that constitutively high expression of GLUT1 improves CAR T cell function against low antigen density tumor cell lines in vitro. FIG. 3A illustrates IFNγ and IL-2 production after 24 hours co-culture with target-specific tumor lines at Day 14 post-activation.

FIG. 3B illustrates frequency of CD69⁺ or indicated cytokine-producing cells after 24-hour stimulation with target cells at Day 15. Gated on total, live CD8+ and CD4+ T cells.

FIG. 3C illustrates GLUT1 versus control GPC2 CART cell lysis of GFP+ SMS-SAN target cells at different effector:target cell ratio. Tumor cell growth was quantified by measuring tumor cell GFP fluorescence using an Incucyte every 3 hours. GFP signal was normalized to the time 0 signal. Data are mean±s.e.m. of triplicate wells. Representative of two independent experiments.

FIG. 3D illustrates GLUT1 versus control GPC2 CART cell lysis of GFP+ NBSD target cells at different effector:target cell ratio. Tumor cell growth was quantified by measuring tumor cell GFP fluorescence using an Incucyte every 3 hours. GFP signal was normalized to the time 0 signal. Data are mean±s.e.m. of triplicate wells. Representative of two independent experiments.

FIG. 3E illustrates GLUT1 versus control GPC2 CART cell lysis of GFP+ NGP− GPC2 target cells at different effector:target cell ratio. Tumor cell growth was quantified by measuring tumor cell GFP fluorescence using an Incucyte every 3 hours. GFP signal was normalized to the time 0 signal. Data are mean±s.e.m. of triplicate wells. Representative of two independent experiments.

FIG. 3F illustrates expression of exhaustion-associated markers by GPC2 cells and GPC2-Glut1 CART cells at Day 15 post-activation.

FIGS. 4A-4C graphically summarize the results of experiments performed to illustrate that GPC2-GLUT1 CAR T cells have higher proliferative potential. FIG. 4A illustrates that Glut1 overexpression has no effect on the proliferation of non-stimulated GPC2 CAR T cells, while both CD4 and CD8 GPC2-Glut1 CAR T cells proliferated at higher rates when GPC2 CAR T cells were stimulated through their CAR with SMS-SAN tumor lines. On Day 14 post-activation GPC2 and GPC2-GLUT1 CAR T cells were loaded with proliferation dye and cells were mixed at 1:1 ratio with SMS-SAN target cells and co-cultured for 96 hrs. CAR T cell proliferation has been followed as a percentage of cells losing the proliferation dye.

FIG. 4B illustrates that Glut1 overexpression has no effect on the proliferation of non-stimulated GPC2 CAR T cells, while both CD4 and CD8 GPC2-Glut1 CAR T cells proliferated at higher rates when GPC2 CAR T cells were stimulated through their CAR with NGP-GPC2 tumor lines. On Day 14 post-activation GPC2 and GPC2-GLUT1 CAR T cells were dyed with proliferation dye and cells were mixed at 1:1 ratio with NGP-GPC2 target cells and co-cultured for 96 hrs. CAR T cell proliferation has been followed as a percentage of cells losing the proliferation dye.

FIG. 4C illustrates that increased proliferation was associated with more CD25+ activated cells.

FIGS. 5A-5D graphically summarize the results of experiments performed to illustrate that co-expression of GLUT1 improves function of exhausted CAR T cells in vivo. FIG. 5A illustrates the experimental scheme of the experiments described in Example 5 (top) and proportion of GLUT1+ CART cells and levels of CAR expression at the day of injection (bottom). NSG mice were inoculated with 1×10⁶ Nalm6-GD2 leukemia cells. On Day 4, 3×10⁶ control or GLUT1 HA-GD2 CAR+ T cells were transferred intravenously.

FIG. 5B illustrates blood sample analysis at days 25 and 40. Enhanced expansion and persistence of Glut1+GD2 CART cells were observed as compared to HA only CAR T cells. Mice receiving HA-GD2-GLUT1 CAR T cells displayed increased peripheral blood T cells on Day 25 and Day 40.

FIG. 5C illustrates tumor growth evaluation using weekly bioluminescent imaging. Mice injected with Glut1+GD2 CAR T cells exhibited complete tumor clearance by Day 19 post-tumor injection (pti), whereas mice receiving GD2 CAR T cells showed no response to the treatment.

FIG. 5D illustrates tumor growth monitored by bioluminescent imaging. Mice injected with Glut1+GD2 CAR T cells exhibited complete tumor clearance by Day 19 post-tumor injection (pti), whereas mice receiving GD2 CAR T cells showed no response to the treatment (left panel). The mice were re-challenged with Nalm6-GD2 leukemia at Day 52 (right, top panel). Four days post-rechallenged (day 64), no signal of tumor could be detected. The mice were re-challenged again (day 60) with Nalm6 or Nalm6-GD2+ tumor line (right, bottom panel). The Glut1+GD2 CAR T cells were still persisting and active against Nalm6-GD2+ tumor cells in mice re-challenged with Nalm6-GD2+, but not in mice re-challenged with Nalm6. Data are mean±s.e.m. of n=5 mice per group. P values determined by unpaired two-tailed t-tests.

FIGS. 6A-6D graphically summarize the results of experiments performed to illustrate that GLUT1 overexpression causes changes at transcriptome level in exhausted CAR T cells. FIG. 6A is a volcano plot representing RNA-sequencing analysis of HA-GD2 and HA-GD2-Glut1 CAR T cells at Day 14 post activation. Transcriptomic profiling of HA-GD2-Glut1 CAR T cells showed that GLUT1 overexpression increases expression levels of genes involved in electron transport chain in mitochondria, which indicates high mitochondrial activity.

FIG. 6B graphically summarizes the results of Gene Set Enrichment Analysis (GSEA) of the most differentially expressed genes. GSEA is a computational method that determines whether an a priori defined set of genes shows statistically significant, concordant differences between two biological states (e.g., phenotypes). More information in this regard can be found at www.gsea-msigdb.org/gsea/index.jsp. A significant upregulation of genes involved in histone modification was observed, suggesting that GLUT1 overexpression induces epigenetic remodeling. Significantly different genes represented as a log of fold change were identified by DESeq2 (Wald test) and are shown in red (FDR≤0.05, n=3 donors).

FIG. 6C is GSEA analysis of the most differentially expressed genes. A significant upregulation of genes involved in histone modification was observed, suggesting that GLUT1 overexpression induces epigenetic remodeling. Significantly different genes represented as a log of fold change were identified by DESeq2 (Wald test) and are shown in red (FDR≤0.05, n=3 donors).

FIG. 6D is GSEA analysis of the most differentially expressed genes. A significant upregulation of genes involved in histone modification was observed, suggesting that GLUT1 overexpression induces epigenetic remodeling. Significantly different genes represented as a log of fold change were identified by DESeq2 (Wald test) and are shown in red (FDR≤0.05, n=3 donors).

FIGS. 7A-7E graphically summarize that Glut1 overexpression increases mitochondrial activity. FIG. 7A is flow cytometry analysis of HA-GD2 and HA-GD2-GLUT1 T cells stained with MitoTracker Green (mitochondrial mass) and MitoTracker Deep Red (mitochondrial membrane potential). Exhausted CAR T cells with overexpressed GLUT1 have increased membrane potential, but there was no difference in mitochondrial mass. At Day 8 post activation CAR T cells were stained with MitoTracker Green (mitochondrial mass) or MitoTracker Deep Red (membrane potential). Histograms of representative donor (top). MFI of mitochondrial mass and membrane potential (n=3 donors) (bottom).

FIG. 7B is flow cytometry analysis of HA-GD2 and HA-GD2-GLUT1 T cells stained with mitoSOX Red dye. GLUT1 overexpression decreased the levels of ROS in mitochondria (n=1 donor).

FIG. 7C is graphical representation of mitochondrial fitness of CAR T cells overexpressing GLUT1 at Day 11 post activation. The results of Mito Stress Test using Seahorse analyzer on exhausted and non-exhausted CAR T cells overexpressing GLUT1 are shown. Higher levels of GLUT1 increased basal glycolysis in GD2-HA CART cells but not in CD1928 cells. Increased basal mitochondrial respiration and ATP linked respiration for both types of CAR T cells were observed, indicating higher mitochondrial activity at the base line. O₂ consumption rate (OCR) and extracellular acidification rate (ECAR) were measured in real-time under basal conditions and in response to mitochondrial inhibitors.

FIG. 7D is a graphical representation of mitochondrial fitness of CAR T cells overexpressing GLUT1 at Day 11 post activation. Basal OCR and ECAR, ATP-linked respiration and spare respiratory capacity (SRC) were calculated. Spare Respiratory Capacity (SRC) was increased as well in GLUT1 overexpressing CAR T cells.

FIG. 7E is a graph illustrating that GLUT1 overexpressing CAR T cells were able to engage effector response to greater extend after antigen encounter. Extracellular acidification rate (ECAR) increased upon CAR T cells activation with 5 μg/ml of idiotype crosslinked with 5 μg/ml goat anti-mouse secondary antibody. Representative of n=2 repeats.

FIGS. 8A-8E graphically summarize GLUT1 overexpressing non-exhausted CAR T cells exhibit increased effector function in vivo. FIG. 8A is a schematic of the experiment design for testing the ability of GLUT1 overexpression to improve non-exhausted CAR T cell effector function in vivo. NSG mice were inoculated with injected IV with 1×10⁶ of SMS-SAN-firefly luciferase (fLuc) neuroblastoma tumor cells. Four days later, mice were injected with 10×10⁶ of CD19bb (negative control), GPC2, or GPC2-GLUT1 overexpressing CAR T cells.

FIG. 8B illustrates T cell concentration in blood at indicated days post tumor injection. Although there was no significant difference in T cell concentration between the groups at Day 15, mice injected with GPC2-GLUT1 exhibited higher number of CD8+ T cells. By Day 29, mice injected with GLUT1 overexpressing CAR T cells had significant more T cells than control group.

FIG. 8C illustrates tumor growth monitored by bioluminescent imaging. Data are mean±s.e.m. of n=5 mice per group. The difference in T cell concentration in mice blood correlated with significantly delayed tumor growth in GPC2-GLUT1 mice group.

FIG. 8D is a schematic of the experiment design for testing the ability of GLUT1 overexpression to improve non-exhausted CAR T cell effector function in vivo. NSG mice were inoculated with 1×10⁶ Nalm6-firefly luciferase (fLuc) tumor cells IV on Day 0. Four days post tumor injection, mice were evaluated and normalized for engraftment using bioluminescent imaging. Five mice per group were injected IV with 1×10⁶ CD1928 CAR, CD1928-Glut1 CAR T cells on Day 4.

FIG. 8E a graph illustrating tumor growth monitored by bioluminescent imaging. Data are mean±s.e.m. of n=5 mice per group. P values determined by 2-way ANOVA tests. Weekly tumor bioluminescent imaging showed that GLUT1 overexpressing CAR T cells significantly delayed tumor growth.

FIGS. 9A-9C graphically summarize that GLUT3 overexpression increases mitochondrial fitness and effector function in vitro and in vivo. FIG. 9A illustrates that GLUT3 overexpression restored mitochondrial membrane potential of HA-GD2 exhausted CAR T cells without changing mitochondrial mass. At Day 8, post activation CAR T cells were stained with MitoTracker Green (mitochondrial mass) or MitoTracker Deep Red (membrane potential). Histograms of representative donor and MFI of mitochondrial mass and membrane potential are shown (n=3 donors).

FIG. 9B illustrates that GLUT3 overexpression augments IL-2 secretion after activation of T cells (mock) upon activation with anti-CD3/CD28 coated beads both exhausted and non-exhausted CAR T cells, HA-GD2 and CD1928 respectively, when stimulated with CAR-specific antigen. IL-2 secretion by mock or CAR T cells stimulated with anti-CD3/CD28 coated beads (3:1 B:T ratio) or Nalm6-GD2 tumor line at Day 14 post activation (n=1-2 donors).

FIG. 9C illustrates that mice injected with HA-GD2-GLUT3 CAR T cells showed significantly lower tumor burden at Day 47 post tumor injection and overall faster tumor clearance depicted as lower area under the curve (AUC).

### DETAILED DESCRIPTION OF THE DISCLOSURE

The present disclosure generally relates to, inter alia, methods and compositions for the prevention and/or treatment of various health conditions. In particular, described herein are immune cells that have been engineered to express elevated levels of one or more glucose transporters, and particularly relate to engineered immune cells exhibiting increased glycolytic flux and/or enhanced effector functions. Also provided are methods for generating a population of engineered immune cells with enhanced effector function, pharmaceutical compositions the same, as well as methods and kits for the prevention and/or treatment of a health condition in subjects in need thereof.

The immune system comprises specialized cell populations that are conditioned to respond rapidly and vigorously to antigenic and inflammatory signals. Emerging data indicate that cellular metabolism regulates immune cell functions and differentiation, and consequently influences the final outcome of the adaptive and innate immune response. In particular, the growth, function, survival, and differentiation of activated immune cells depend on dramatic increases in glucose metabolism as fuel, a process that is directly regulated and has a profound impact on health and disease.

In particular, it has been reported that the functions of peripheral T cells are maintained and are intimately linked to metabolism. Specific effector functions are unable to proceed without the cell adopting the appropriate metabolic state. T cells rapidly transition between resting catabolic states (naive and memory T cells) to one of growth and proliferation (effector T cells) during normal immune responses. Without being bound to any particular theory, it is believed that the commitment of an immune cell to a specific metabolic pathway depends on the particular function. This is evident in the subsets of CD4⁺T cells where effector T cells and Th17 cells rely on aerobic glycolysis, while memory T cells and T regulatory cells (Treg) rely on fatty acid oxidation to produce energy. Aerobic glycolysis is also utilized for energy by activated dendritic cells, neutrophils, and pro-inflammatory macrophages. Recent reports have indicated that T cell activation is not accompanied merely by a switch from oxidative metabolism to glycolysis, but that both pathways are upregulated to support bioenergetics demands. This intimate interrelationship between T cell activation and metabolism led to the concept that changes in T cell metabolism are not simply a consequence of antigen-induced activation, but rather a parameter that determines T cell proliferation and fate decision. Additional information in this regard can be found in, for example, a recent review by Palmer et al. (Glucose metabolism regulates T cell activation, differentiation, and functions. Front. Immunol. Vol. 6; 22 Jan. 2015), which is herein incorporated by reference.

As described in greater detail below, experimental data presented herein demonstrate that by overexpression of a glucose transporter, for example GLUT1, in immune cells increases glycolytic flux and leads to enhanced mitochondrial activity, increased immune cell activation, cytokine secretion, and tumor killing. Moreover, overexpression of glucose transporters can improve CAR T cell effector function even in the context of T cell exhaustion, a phenomenon whereby excessive signaling leads to diminished T cell function and ultimately T cell failure and death. In principle, this approach provides a new approach to engineer immune cells for enhanced metabolic reprogramming to overcome the nutrient depleted microenvironment present in cancer. Immune cells expressing natural receptors or those engineered to express antigen specific receptors such as chimeric antigen receptors (CARs), recombinant TCRs or others can be metabolically reprogrammed by overexpression of glucose transporters in order to improve their cytotoxic function, proliferation and in vivo persistence.

In addition, engineering immune cells by overexpressing one or more glucose transporters to regulate glycolytic flux can increase their effector function and expansion. This approach could be of important value in the context of adoptive immunotherapy, where a specific receptor engagement is required such as in CAR therapy (including T cells, NK cells and NK T cells), TCR-modified T cell or TILs and where tumor-reactive cells compete for nutrients with tumor cells. In particular, the approach described herein may be particularly valuable for the treatment of solid tumors where a hostile TME with limited nutrients is documented. In addition, this approach could be applied to increase proliferation and expansion of immune cell products throughout the manufacturing process.

### DEFINITIONS

Unless otherwise defined, all terms of art, notations and other scientific terms or terminology used herein are intended to have the meanings commonly understood by those of skill in the art to which this disclosure pertains. In some cases, terms with commonly understood meanings are defined herein for clarity and/or for ready reference, and the inclusion of such definitions herein should not necessarily be construed to represent a substantial difference over what is generally understood in the art. Many of the techniques and procedures described or referenced herein are well understood and commonly employed using conventional methodology by those skilled in the art.

The singular form “a”, “an”, and “the” include plural references unless the context clearly dictates otherwise. For example, the term “a cell” includes one or more cells, comprising mixtures thereof. “A and/or B” is used herein to include all of the following alternatives: “A”, “B”, “A or B”, and “A and B”.

The term “about”, as used herein, has its ordinary meaning of approximately. If the degree of approximation is not otherwise clear from the context, “about” means either within plus or minus 10% of the provided value, or rounded to the nearest significant figure, in all cases inclusive of the provided value. Where ranges are provided, they are inclusive of the boundary values.

The terms “administration” and “administering”, as used herein, refer to the delivery of a bioactive composition or formulation by an administration route comprising, but not limited to, oral, intravenous, intra-arterial, intramuscular, intraperitoneal, subcutaneous, intramuscular, and topical administration, or combinations thereof. The term includes, but is not limited to, administering by a medical professional and self-administering.

“Cancer” refers to the presence of cells possessing several characteristics of cancer-causing cells, such as uncontrolled proliferation, immortality, metastatic potential, rapid growth and proliferation rate, and certain characteristic morphological features. Cancer cells can aggregate into a mass, such as a tumor, or can exist alone within a subject. A tumor can be a solid tumor, a soft tissue tumor, or a metastatic lesion. As used herein, the term “cancer” also encompasses other types of non-tumor cancers. Non-limiting examples include blood cancers or hematological cancers, such as leukemia. Cancer can include premalignant, as well as malignant cancers.

The terms “cell”, “cell culture”, and “cell line” refer not only to the particular subject cell, cell culture, or cell line but also to the progeny or potential progeny of such a cell, cell culture, or cell line, without regard to the number of transfers or passages in culture. It should be understood that not all progeny are exactly identical to the parental cell. This is because certain modifications may occur in succeeding generations due to either mutation (e.g., deliberate or inadvertent mutations) or environmental influences (e.g., methylation or other epigenetic modifications), such that progeny may not, in fact, be identical to the parent cell, but are still included within the scope of the term as used herein, so long as the progeny retain the same functionality as that of the original cell, cell culture, or cell line.

The term “operably linked”, as used herein, denotes a physical or functional linkage between two or more elements, e.g., polypeptide sequences or polynucleotide sequences, which permits them to operate in their intended fashion. For example, the term “operably linked” when used in context of the nucleic acid molecules described herein or the coding sequences and promoter sequences in a nucleic acid molecule means that the coding sequences and promoter sequences are in-frame and in proper spatial and distance away to permit the effects of the respective binding by transcription factors or RNA polymerase on transcription. It should be understood that, operably linked elements may be contiguous or non-contiguous (e.g., linked to one another through a linker). In the context of polypeptide constructs, “operably linked” refers to a physical linkage (e.g., directly or indirectly linked) between amino acid sequences (e.g., different segments, portions, regions, or domains) to provide for a described activity of the constructs. Operably linked segments, portions, regions, and domains of the polypeptides or nucleic acid molecules disclosed herein may be contiguous or non-contiguous (e.g., linked to one another through a linker).

The term “recombinant” or “engineered” nucleic acid molecule, polypeptide, or cell as used herein, refers to a nucleic acid molecule, polypeptide, or cell that has been altered through human intervention.

As used herein, and unless otherwise specified, a “therapeutically effective amount” or a “therapeutically effective number” of an agent is an amount or number sufficient to provide a therapeutic benefit in the treatment or management of a disease, e.g., cancer, or to delay or minimize one or more symptoms associated with the disease. A therapeutically effective amount or number of a compound means an amount or number of therapeutic agent, alone or in combination with other therapeutic agents, which provides a therapeutic benefit in the treatment or management of the disease. The term “therapeutically effective amount” can encompass an amount or number that improves overall therapy of the disease, reduces or avoids symptoms or causes of the disease, or enhances therapeutic efficacy of another therapeutic agent. An example of an “effective amount” is an amount sufficient to contribute to the treatment, prevention, or reduction of a symptom or symptoms of a disease, which could also be referred to as a “therapeutically effective amount.” A “reduction” of a symptom means decreasing of the severity or frequency of the symptom(s), or elimination of the symptom(s). The exact amount of a composition including a “therapeutically effective amount” will depend on the purpose of the treatment, and will be ascertainable by one skilled in the art using known techniques (see, e.g., Lieberman, Pharmaceutical Dosage Forms (vols. 1-3, 2010); Lloyd, The Art, Science and Technology of Pharmaceutical Compounding (2016); Pickar, Dosage Calculations (2012); and Remington: The Science and Practice of Pharmacy, 22nd Edition, 2012, Gennaro, Ed., Lippincott, Williams & Wilkins).

As used herein, a “subject” or an “individual” includes animals, such as human (e.g., human subject) and non-human animals. In some embodiments, a “subject” or “individual” is a patient under the care of a physician. Thus, the subject can be a human patient or a subject who has, is at risk of having, or is suspected of having a disease of interest (e.g., cancer) and/or one or more symptoms of the disease. The subject can also be a subject who is diagnosed with a risk of the condition of interest at the time of diagnosis or later. The term “non-human animals” includes all vertebrates, e.g., mammals, e.g., rodents, e.g., mice, non-human primates, and other mammals, such as e.g., sheep, dogs, cows, chickens, and non-mammals, such as amphibians, reptiles, etc.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges, and are also encompassed within the disclosure, subject to any specifically excluded limit in the stated range. Where the stated range includes one or both of the limits, ranges excluding either or both of those included limits are also included in the disclosure.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

It is understood that aspects and embodiments of the disclosure described herein include “comprising,” “consisting,” and “consisting essentially of” aspects and embodiments. As used herein, “comprising” is synonymous with “including”, “containing”, or “characterized by”, and is inclusive or open-ended and does not exclude additional, unrecited elements or method steps. As used herein, “consisting of” excludes any elements, steps, or ingredients not specified in the claimed composition or method. As used herein, “consisting essentially of” does not exclude materials or steps that do not materially affect the basic and novel characteristics of the claimed composition or method. Any recitation herein of the term “comprising”, particularly in a description of components of a composition or in a description of steps of a method, is understood to encompass those compositions and methods consisting essentially of and consisting of the recited components or steps.

Headings, e.g., (a), (b), (i) etc., are presented merely for ease of reading the specification and claims. The use of headings in the specification or claims does not require the steps or elements be performed in alphabetical or numerical order or the order in which they are presented.

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable sub-combination. All combinations of the embodiments pertaining to the disclosure are specifically embraced by the present disclosure and are disclosed herein just as if each and every combination was individually and explicitly disclosed. In addition, all sub-combinations of the various embodiments and elements thereof are also specifically embraced by the present disclosure and are disclosed herein just as if each and every such sub-combination was individually and explicitly disclosed herein.

### GLUCOSE TRANSPORT IN REGULATION OF IMMUNE CELL ACTIVATION

Glucose transporters (GLUTs) are integral membrane proteins that contain 12 membrane-spanning helices with both the amino and carboxyl termini exposed on the cytoplasmic side of the plasma membrane, and facilitate the transport of glucose across the plasma membrane, a process known as facilitated diffusion. To date, there are 14 mammalian facilitative glucose transporters, and they belong to the sugar transporter family in the major facilitator superfamily (MFS). They are encoded by the solute linked carrier family 2, subfamily A gene family, SLC2A. It has been reported that many of these proteins have substrates other than hexoses. Glucose transporters are categorized into three classes based on their protein sequence and structural similarity. Class I, the “glucose transporters”, includes GLUT1, GLUT2, GLUT3, GLUT4, and GLUT14. Class II glucose transporters include GLUT5, GLUT7, GLUT9, and GLUT11, and are known to transport fructose as well. Class III is made up of GLUT6, GLUT8, GLUT10, GLUT12, and GLUT13 (HMIT1); this class is believed to be the most ancient group. Additional information regarding glucose transporters in mammalian cells can be found in, for examples, a recent review by Long W. and Cheeseman C. (Structure of, and functional insight into the GLUT family of membrane transporter. Dove Press, 6 Oct. 2015 Volume 2015:7 pp. 167-183), which is herein incorporated by reference.

GLUTs share various mechanisms of regulation, for example in response to HIF-1α or p53. However, they often respond to many distinct stimuli. For example, GLUT1 has been found to be activated as a response to HIF-1α in glioblastoma stem cells. Additionally, other hypoxia-associated factors, such as VEGF receptor and calcium channel transactivation, were described to upregulate GLUT1 synthesis and trafficking to the cellular membrane.

Epigenetic studies have contributed a better understanding of glucose transporter regulation. For example, in the mouse brain, it was observed that fasting-induced production of the ketone body β-hydroxybutyrate enhances expression of GLUT1. This enhanced expression of GLUT1 is correlated with an increase of H3K9 acetylation at an important cis-regulatory region of the GLUT1 gene. It was also reported that CRISPR/Cas9-mediated disruption of HDAC2 increases GLUT1 expression.

It has been reported that immune cell activation must be controlled to allow proper immunity while preventing inappropriate inflammatory immune responses. In particular, glucose metabolism is an important element in supporting immune cell growth, proliferation, and effector function. For example, glucose transporters, such as GLUT1, and glycolysis are upregulated upon activation and differentiation of CD4 T cells into effectors (e.g., Teff, Th1, Th2, and Th17). Regulatory T cells (Treg), however, express lower levels of GLUT1 and utilize lipid oxidation rather than glycolysis as a primary metabolic program. Importantly, cell metabolism must match the demands of each cell type and the inhibition of glucose metabolism prevents specification and function of Teff, while Treg are preferentially generated if glucose is limiting or glycolysis is inhibited.

To acquire a fully activated state upon antigen encounter, T cells must undergo metabolic reprogramming to support cell growth, proliferation and cytotoxic function. This reprogramming is sensitive to cell intrinsic (such as regulation of nutrient transporters) and extrinsic (such as nutrient availability) factors. The tumor microenvironment in cancer is associated with limited nutrient availability wherein both the tumor cells and infiltrating immune cells compete for limiting nutrients. Inadequate metabolic reprogramming and resulting dysfunction of tumor infiltrating lymphocytes is an important contributor to immune evasion in cancer limits the potency of cancer immunotherapies, especially for solid tumors. Manipulation of T cell metabolism, therefore, may provide a new approach to modulate immunity and reduce Teff function in inflammatory and autoimmune diseases. It is unclear, however, how T cell metabolism is regulated and what impact disruption of glucose metabolism may have in vivo, where a wide variety of alternate nutrients are available to potentially replace glucose.

Experimental data presented herein demonstrate that by overexpression of a glucose transporter, for example GLUT1, in immune cells increases glycolytic flux and leads to enhanced immune cell activation, cytokine secretion and tumor killing. Moreover, overexpression of glucose transporters can improve CAR T cell effector function even in the context of T cell exhaustion, a phenomenon whereby excessive signaling leads to diminished T cell function and ultimately T cell failure and death. In principle, this approach provides a new approach to engineer immune cells for enhanced metabolic reprogramming to overcome the nutrient depleted microenvironment present in cancer. Immune cells expressing natural receptors or those engineered to express antigen specific receptors such as chimeric antigen receptors (CARs), recombinant TCRs or others can be metabolically reprogrammed by overexpression of glucose transporters in order to improve their cytotoxic function, proliferation and in vivo persistence.

In addition, engineering immune cells by overexpressing one or more glucose transporters to regulate glycolytic flux can increase their effector function and expansion. This approach could be of great value in the context of adoptive immunotherapy, where a specific receptor engagement is required such as in CAR therapy (including T cells, NK cells and NK T cells), TCR-modified T cell or TILs and where tumor-reactive cells compete for nutrients with tumor cells. In particular, the approach described herein may be particularly valuable for the treatment of solid tumors where a hostile TME with limited nutrients is documented. In addition, this approach could be applied to increase proliferation and expansion of immune cell products throughout the manufacturing process.

### METHODS FOR GENERATING ENGINEERED IMMUNE CELLS WITH ENHANCED EFFECTOR FUNCTIONS

As described in greater detail herein, some embodiments of the present disclosure provide various methods for generating an engineered immune cell with enhanced effector function, the method comprising introducing into the immune cell a nucleic acid and/or a polypeptide capable of conferring increased expression of one or more glucose transporters in the immune cell. Non-limiting exemplary embodiments of the disclosed methods can include one or more of the following features. In some embodiments, the introduced nucleic acid and/or polypeptide confers an increase in expression of one or more GLUT genes in the engineered immune cell by at least 10%, such as at least about 10%, at least about 20%, at least about 30%, at least about 40%, at least about 50%, at least about 60%, at least about 70%, at least about 80%, at least about 90%, at least about 2 times, about three times, about four time, about five times, about six times, about seven times, about eight times, about nine times, about 20 times, about 50 times, about 100 times, or about 200 times compared to a reference immune cell. In some embodiments, the reference immune cell is an immune cell that does not include the same nucleic acid and/or polypeptide.

In some embodiments, the method comprising introducing into the immune cell a nucleic acid and/or a polypeptide that results in increased expression of one or more endogenous genes encoding glucose transporter in the immune cell. In some embodiments, the introduced nucleic acid and/or a polypeptide results in an increase of H3K9 acetylation in the cis-regulatory region of the GLUT1 gene. In some embodiments, the introduced nucleic acid and/or a polypeptide results in down-regulation of HDAC2. In some embodiments, the introduced nucleic acid comprises an antisense oligonucleotide targeting HDAC2.

In some embodiments, the introduced nucleic acid includes a sequence encoding one or more glucose transporters, or a functional variant thereof. One skilled in the art will readily understand that the term “functional variant thereof” refers to a molecule having qualitative biological activity in common with the wild-type molecule from which the functional variant was derived. For example, when referencing a polypeptide having an enzymatic activity (e.g., an enzyme such as a glucose transporter), the term functional variant of an enzyme refers to enzymes that have a polypeptide sequence having at least about 70%, at least about 75%, at least about 80%, at least about 85%, at least about 90%, at least about 95% or at least about 99% identical to a polypeptide sequence encoding the enzyme. In some embodiments, the functional GLUT variants of the disclosure may retain amino acids residues that are recognized as conserved for the enzyme, and may have non-conserved amino acid residues substituted or found to be of a different amino acid, or amino acid(s) inserted or deleted, but which does not affect or has insignificant effect its enzymatic activity, as compared to the enzyme described herein. In some embodiments, the functional GLUT variants of the disclosure have an enzymatic activity that is identical or essentially identical to the biological activity of the wild-type glucose transporter (e.g., glucose transporter) described herein. One skilled in the art will appreciate that functional GLUT variants may be found in nature, i.e. naturally occurring, or be an engineered mutant thereof.

Suitable glucose transporters include, but are not limited to, glucose transporters belonging to Classes I-IV transporters. In some embodiments, the introduced nucleic acid includes a sequence encoding one or more Class I glucose transporters, or a functional variant thereof. In some embodiments, the introduced nucleic acid includes a sequence encoding one or more Class II glucose transporters, or a functional variant thereof. In some embodiments, the introduced nucleic acid includes a sequence encoding one or more Class III glucose transporters, or a functional variant thereof. In some embodiments, the introduced nucleic acid includes a sequence encoding one or more Class IV glucose transporters, or a functional variant thereof. Suitable glucose transporters include, but are not limited to, GLUT1, GLUT2, GLUT3, GLUT4, GLUT6, GLUT8, GLUT9, GLUT10, GLUT11, and GLUT12, or a functional variant thereof.

In some embodiments, the introduced nucleic acid includes a sequence encoding one or more glucose transporters selected from the group consisting of GLUT1, GLUT2, GLUT3, GLUT4, and functional variants of any thereof. In some embodiments, the introduced nucleic acid includes a sequence encoding GLUT1 or a functional variant thereof. In some embodiments, the introduced nucleic acid includes a sequence encoding GLUT2 or a functional variant thereof. In some embodiments, the introduced nucleic acid includes a sequence encoding GLUT3 or a functional variant thereof. In some embodiments, the introduced nucleic acid includes a sequence encoding GLUT4 or a functional variant thereof.

The basic techniques for operably linking two or more sequences of DNA together are familiar to the skilled worker, and such methods have been described in a number of texts for standard molecular biological manipulation. The molecular techniques and methods by which these nucleic acid molecules can be constructed and characterized are described more fully below and in the Examples herein.

In some embodiments of the disclosure, the coding sequence of the one or more glucose transporters is operably linked to a heterologous nucleic acid sequence. In some embodiments, the heterologous nucleic acid sequence comprises a transcription control element or a coding sequence for a selectable marker. In some embodiments, the coding sequence of the one or more glucose transporters is operably linked to a transcription control element. In some embodiments, the transcription control element is a promoter sequence. A non-limiting exemplification of suitable promoter is the immediate early cytomegalovirus (CMV) promoter sequence. This promoter sequence is a strong constitutive promoter sequence capable of driving high levels of expression of any polynucleotide sequence operatively linked thereto. However, other constitutive promoter sequences may also be used, including, but not limited to the simian virus 40 (SV40) early promoter, MoMuLV promoter, an avian leukemia virus promoter, an Epstein-Barr virus immediate early promoter, mouse mammary tumor virus (MMTV), human immunodeficiency virus (HIV) long terminal repeat (LTR) promoter, a Rous sarcoma virus promoter, the elongation factor-la promoter, as well as human gene promoters such as, but not limited to, the actin promoter, the myosin promoter, the hemoglobin promoter, ubiquitin C (UBC) promoter, eIF4A1 promoter, and the creatine kinase promoter. Further, the disclosure should not be limited to the use of constitutive promoters. Inducible promoters are also contemplated as part of the disclosure. The use of an inducible promoter provides a molecular switch capable of turning on expression of the polynucleotide sequence which it is operatively linked when such expression is desired, or turning off the expression when expression is not desired. Examples of inducible promoters include, but are not limited to a metallothionine promoter, a glucocorticoid promoter, a progesterone promoter, radiation-inducible promoters, hypoxia-inducible promoter, ROS-inducible promoter, and a tetracycline promoter.

In some embodiments, the nucleic acid sequence encoding one or more glucose transporters can be incorporated into an expression cassette or an expression vector. It will be understood that an expression cassette generally includes a construct of genetic material that contains coding sequences and enough regulatory information to direct proper transcription and/or translation of the coding sequences in a cell, in vivo and/or ex vivo. Generally, the expression cassette may be inserted into a vector for targeting to a desired host cell and/or into a desired host cell and/or into an individual. As such, in some embodiments, an expression cassette of the disclosure include a nucleic acid sequence encoding one or more glucose transporters as disclosed herein, which is operably linked to expression control elements, such as a promoter, and optionally, any or a combination of other nucleic acid sequences that affect the transcription or translation of the coding sequence. An expression cassette can be inserted into a plasmid, cosmid, virus, autonomously replicating polynucleotide molecule, phage, as a linear or circular, single-stranded or double-stranded, DNA or RNA polynucleotide molecule, derived from any source, capable of genomic integration or autonomous replication, including a nucleic acid molecule where one or more nucleic acid sequences has been linked in a functionally operative manner, i.e., operably linked.

Also provided herein are vectors, plasmids, or viruses containing one or more of the nucleic acid molecules encoding any glucose transporters as described herein. The nucleic acid molecules can be contained within a vector that is capable of directing their expression in, for example, a cell that has been transformed/transduced with the vector. Suitable vectors for use in eukaryotic and prokaryotic cells are known in the art and are commercially available, or readily prepared by a skilled artisan. See for example, Sambrook, J., & Russell, D. W. (2012). Molecular Cloning: A Laboratory Manual (4th ed.). Cold Spring Harbor, N.Y.: Cold Spring Harbor Laboratory and Sambrook, J., & Russel, D. W. (2001). Molecular Cloning: A Laboratory Manual (3rd ed.). Cold Spring Harbor, N.Y.: Cold Spring Harbor Laboratory (jointly referred to herein as “Sambrook”); Ausubel, F. M. (1987). Current Protocols in Molecular Biology. New York, N.Y.: Wiley (including supplements through 2014); Bollag, D. M. et al. (1996). Protein Methods. New York, N.Y.: Wiley-Liss; Huang, L. et al. (2005). Nonviral Vectors for Gene Therapy. San Diego: Academic Press; Kaplitt, M. G. et al. (1995). Viral Vectors: Gene Therapy and Neuroscience Applications. San Diego, Calif.: Academic Press; Lefkovits, I. (1997). The Immunology Methods Manual: The Comprehensive Sourcebook of Techniques. San Diego, Calif.: Academic Press; Doyle, A. et al. (1998). Cell and Tissue Culture: Laboratory Procedures in Biotechnology. New York, N.Y.: Wiley; Mullis, K. B., Ferre, F. & Gibbs, R. (1994). PCR: The Polymerase Chain Reaction. Boston: Birkhauser Publisher; Greenfield, E. A. (2014). Antibodies: A Laboratory Manual (2nd ed.). New York, N.Y.: Cold Spring Harbor Laboratory Press; Beaucage, S. L. et al. (2000). Current Protocols in Nucleic Acid Chemistry. New York, N.Y.: Wiley, (including supplements through 2014); and Makrides, S. C. (2003). Gene Transfer and Expression in Mammalian Cells. Amsterdam, NL: Elsevier Sciences B.V., the disclosures of which are incorporated herein by reference).

DNA vectors can be introduced into eukaryotic cells via conventional transformation or transfection techniques. Suitable methods for transforming or transfecting cells can be found in Sambrook et al. (2012, supra) and other standard molecular biology laboratory manuals, such as, calcium phosphate transfection, DEAE-dextran mediated transfection, transfection, microinjection, cationic lipid-mediated transfection, electroporation, transduction, scrape loading, ballistic introduction, nucleoporation, hydrodynamic shock, and infection.

Viral vectors that can be used in the disclosure include, for example, retrovirus vectors, adenovirus vectors, and adeno-associated virus vectors, lentivirus vectors, herpes virus, simian virus 40 (SV40), and bovine papilloma virus vectors (see, for example, Gluzman (Ed.), Eukaryotic Viral Vectors, CSH Laboratory Press, Cold Spring Harbor, N.Y.). For example, a chimeric receptor as disclosed herein can be produced in a eukaryotic cell, such as a mammalian cell (e.g., COS cells, NIH 3T3 cells, or HeLa cells). These cells are available from many sources, including the American Type Culture Collection (Manassas, Va.). In selecting an expression system, care should be taken to ensure that the components are compatible with one another. Artisans or ordinary skill are able to make such a determination. Furthermore, if guidance is required in selecting an expression system, skilled artisans may consult P. Jones, “Vectors: Cloning Applications”, John Wiley and Sons, New York, N.Y., 2009). Accordingly, the nucleic acid sequence encoding one or more glucose transporters can be incorporated into a viral vector. In some embodiments, the vector is a viral vector derived from a lentivirus, an adeno-virus, an adeno-associated virus, a baculovirus, or a retrovirus. In some embodiments, the nucleic acid is incorporated into a nucleic construct for use in guide RNA-directed CRISPR-mediated knock-in procedure, CRISPR/Cas9 genome editing, or DNA-guided endonuclease genome editing with NgAgo (Natronobacterium gregoryi Argonaute), or TALENs genome editing (transcription activator-like effector nucleases).

The nucleic acid molecules provided can contain naturally occurring sequences, or sequences that differ from those that occur naturally, but, due to the degeneracy of the genetic code, encode the same polypeptide, e.g., antibody. These nucleic acid molecules can consist of RNA or DNA (for example, genomic DNA, cDNA, or synthetic DNA, such as that produced by phosphoramidite-based synthesis), or combinations or modifications of the nucleotides within these types of nucleic acids. In addition, the nucleic acid molecules can be double-stranded or single-stranded (e.g., either a sense or an antisense strand).

The nucleic acid molecules are not limited to sequences that encode polypeptides (e.g., antibodies); some or all of the non-coding sequences that lie upstream or downstream from a coding sequence (e.g., the coding sequence of a chimeric receptor) can also be included. Those of ordinary skill in the art of molecular biology are familiar with routine procedures for isolating nucleic acid molecules. They can, for example, be generated by treatment of genomic DNA with restriction endonucleases, or by performance of the polymerase chain reaction (PCR). In the event the nucleic acid molecule is a ribonucleic acid (RNA), molecules can be produced, for example, by in vitro transcription.

In some embodiments, the immune cell is T lymphocyte, a natural killer (NK) cell, or a natural killer T cell (NKT). In some embodiments, the T lymphocyte is a CD8+ T cytotoxic lymphocyte cell selected from the group consisting of naïve CD8+ T cells, central memory CD8+ T cells, effector memory CD8+ T cells, effector CD8+ T cells, CD8+ stem memory T cells, bulk CD8+ T cells. In some embodiments, the lymphocyte is a CD4+ T helper lymphocyte cell selected from the group consisting of naïve CD4+ T cells, central memory CD4+ T cells, effector memory CD4+ T cells, effector CD4+ T cells, CD4+ stem memory T cells, and bulk CD4+ T cells. In some embodiments, the immune cell is ex vivo. In some embodiments, the immune cell is in vitro. In some embodiments, the immune cell is in vivo. In some embodiments, the immune cell is an animal cell. In some embodiments, the animal cell is a mammalian cell. In some embodiments, the animal cell is a mouse cell. In some embodiments, the animal cell is a human cell. In some embodiments, the cell is a non-human primate cell. In some embodiments, the immune cell is obtained by leukapheresis performed on a sample obtained from a subject.

In some embodiments, the methods of the disclosure further include introducing into the immune cells one or more recombinant immune receptors, such as, such as a chimeric antigen receptor (CAR) or a T cell receptor (TCR), and/or nucleic acids encoding the same. For example, the immune cells can include and/or express an antigen-specific receptor, e.g., a receptor that can immunologically recognize and/or specifically bind to an antigen, or an epitope thereof, such that binding of the antigen-specific receptor to antigen, or the epitope thereof, elicits an immune response. In some embodiments, the antigen-specific receptor has antigenic specificity for a cancer antigen, such as a tumor-specific antigen (TSA) or a tumor-associated antigen (TAA).

In some embodiments, the antigen-specific receptor is a T-cell receptor (TCR). A TCR generally comprises two polypeptides (e.g., polypeptide chains), such as an a-chain of a TCR, a β-chain of a TCR, a γ-chain of a TCR, a δ-chain of a TCR, or a combination thereof. Such polypeptide chains of TCRs are known in the art. The antigen-specific TCR can include any amino acid sequence, provided that the TCR can specifically bind to and/or immunologically recognize an antigen, such as a cancer antigen or epitope thereof. In some embodiments, the TCR is an endogenous TCR, e.g., a TCR that is endogenous or native to (naturally-occurring) the T cell. In such a case, the T cell expressing the endogenous TCR can be a T cell that was isolated from a mammal which is known to express the particular cancer antigen. For example, in some embodiments, the T cell is a primary T cell isolated from a mammal having a cancer. In some embodiments, the T cell is a TIL or a T cell isolated from a human cancer patient.

In some embodiments, the immune cells include and/or express a chimeric antigen receptor (CAR). Generally, a CAR includes an antigen binding domain, e.g., a single-chain variable fragment (scFv) of an antibody, fused to a transmembrane domain and an intracellular domain. In this case, the antigenic specificity of a CAR can be encoded by a scFv which specifically binds to the antigen, or an epitope thereof. CARs, and methods of making them, are known in the art.

In some embodiments, the immune cells include one or more nucleic acids encoding an exogenous (e.g., recombinant) antigen-specific receptor. In some embodiments, such exogenous antigen-specific receptors, e.g., exogenous TCRs and CARs can confer specificity for additional antigens to the T cell beyond the antigens for which the endogenous TCR is naturally specific.

In some embodiments, the increased expression of the one or more glucose transporters results in an improved function of non-exhausted CAR T cells, as indicated by for example increased production of interferon gamma (IFNγ), tumor-necrosis factor alpha (TNFα), and/or interleukin-2 (IL-2) relative to the production of these molecules in reference control cells, e.g., cells with native expression levels of glucose transporters. In some embodiments, the increased expression of the one or more glucose transporters results improved function of exhausted CAR T cells. In some embodiments, the increased expression of the one or more glucose transporters results in higher proliferative potential of CAR T cells.

In one aspect, provided herein are immune cells that have been engineered to overexpress one or more glucose transporters. In particular, described herein are immune cells that have been engineered to express elevated levels of one or more glucose transporters, and particularly relate to engineered immune cells exhibiting increased glycolytic flux and/or enhanced effector functions. Some embodiments of the disclosure provide engineered immune cells that have been produced by a method described herein. In some embodiments, the immune cells are in vitro. In some embodiments, the immune cells are ex vivo. In some embodiments, the immune cells are in vivo. In some embodiments, the immune cell is an exhausted immune cell or a non-exhausted immune cell.

In another aspect, provided herein are cell cultures including at least one recombinant cell as disclosed herein, and a culture medium. Generally, the culture medium can be any suitable culture medium for culturing the cells described herein. Techniques for transforming a wide variety of the above-mentioned host cells and species are known in the art and described in the technical and scientific literature. Accordingly, cell cultures including at least one engineered cell as disclosed herein and a culture medium are also within the scope of this application. Methods and systems suitable for generating and maintaining cell cultures are known in the art.

### COMPOSITIONS OF THE DISCLOSURE

### Pharmaceutical Compositions

The engineered immune cells and nucleic acids of the disclosure can be incorporated into compositions, including pharmaceutical compositions. Such compositions generally can include one or more engineered immune cells and nucleic acids of the disclosure and a pharmaceutically acceptable excipient, e.g., a carrier. Accordingly, in one aspect, some embodiments of the disclosure relate to pharmaceutical compositions including a pharmaceutically acceptable excipient and a) an engineered cell of the disclosure; and/or b) a nucleic acid comprising a sequence encoding one or more glucose transporters selected from the group consisting of GLUT1, GLUT2, GLUT3, GLUT4, GLUT6, GLUT8, GLUT9, GLUT10, GLUT11, GLUT12,and functional variants of any thereof.

In some embodiments, the pharmaceutical compositions of the disclosure are formulated for the treating, ameliorating a health condition, e.g., a proliferative disease such as cancer, or for reducing or delaying the onset of the disease.

Non-limiting exemplary embodiments of the pharmaceutical compositions described herein can include one or more of the following features. In some embodiments, the composition includes a nucleic acid encoding one or more glucose transporters, and a pharmaceutically acceptable excipient. In some embodiments, the nucleic acid is encapsulated in a viral capsid or a lipid nanoparticle. In some embodiments, the nucleic acid is incorporated into an expression cassette or an expression vector. In some embodiments, the expression vector is a viral vector. In some embodiments, the viral vector is a lentiviral vector, an adenovirus vector, an adeno-associated virus vector, or a retroviral vector.

In some embodiments, the nucleic acid can be introduced into a host immune cell, for example, a T lymphocyte, an NK cell, or a NKT cell, to produce a recombinant immune cell containing the nucleic acid. In some embodiments, the nucleic acid can be administered into a subject in need thereof.

Introduction of the nucleic acids of the disclosure into cells can be achieved by methods known to those skilled in the art such as, for example, viral infection, transfection, conjugation, protoplast fusion, lipofection, electroporation, nucleofection, calcium phosphate precipitation, polyethyleneimine (PEI)-mediated transfection, DEAE-dextran mediated transfection, liposome-mediated transfection, particle gun technology, calcium phosphate precipitation, direct micro-injection, nanoparticle-mediated nucleic acid delivery, and the like.

Accordingly, in some embodiments, the nucleic acid molecules can be delivered by viral or non-viral delivery vehicles known in the art. For example, the nucleic acid molecule can be stably integrated in the host genome, or can be episomally replicating, or present in the host cell as a mini-circle expression vector for transient expression. Accordingly, in some embodiments, the nucleic acid molecule is maintained and replicated in the host cell as an episomal unit. In some embodiments, the nucleic acid molecule is stably integrated into the genome of the host cell. Stable integration can be achieved using classical random genomic recombination techniques or with more precise techniques such as guide RNA-directed CRISPR/Cas9 genome editing, or DNA-guided endonuclease genome editing with NgAgo (Natronobacterium gregoryi Argonaute), or TALENs genome editing (transcription activator-like effector nucleases). In some embodiments, the nucleic acid molecule is present in the host cell as a mini-circle expression vector for transient expression.

The nucleic acid molecules can be encapsulated in a viral capsid, or a liposome, or a lipid nanoparticle (LNP), or can be delivered by viral or non-viral delivery means and methods known in the art, such as electroporation. For example, introduction of nucleic acids into cells may be achieved by viral transduction. In a non-limiting example, adeno-associated virus (AAV) is engineered to deliver nucleic acids to target cells via viral transduction. Several AAV serotypes have been described, and all of the known serotypes can infect cells from multiple diverse tissue types. AAV is capable of transducing a wide range of species and tissues in vivo with no evidence of toxicity, and it generates relatively mild innate and adaptive immune responses.

Lentiviral-derived vector systems are also useful for nucleic acid delivery and gene therapy via viral transduction. Lentiviral vectors offer several attractive properties as gene-delivery vehicles, including: (i) sustained gene delivery through stable vector integration into host genome; (ii) the capability of infecting both dividing and non-dividing cells; (iii) broad tissue tropisms, including important gene- and cell-therapy-target cell types; (iv) no expression of viral proteins after vector transduction; (v) the ability to deliver complex genetic elements, such as polycistronic or intron-containing sequences; (vi) a potentially safer integration site profile; and (vii) a relatively easy system for vector manipulation and production.

In some embodiments, the composition includes at least one engineered immune cell of the disclosure, and a pharmaceutically acceptable excipient. In some embodiments, the at least one engineered immune cell exhibits an enhanced effector function when introduced into a subject. Examples of effector functions that are enhanced in the engineered immune cells include, but are not limited to growth rate (proliferation), death rate, death rate type, target cell inhibition (cytotoxicity), target cell killing, target cell survival, cluster of differentiation change, macrophage activation, B cell activation, cytokine production, in vivo persistence, and increased glycolytic flux.

In certain embodiments, the pharmaceutical compositions in accordance with some embodiments disclosed herein include cultures of engineered immune cells that can be washed, treated, combined, supplemented, or otherwise altered prior to administration to an individual in need thereof. Furthermore, administration can be at varied doses, time intervals or in multiple administrations.

The pharmaceutical compositions provided herein can be in any form that allows for the composition to be administered to a subject. In some specific embodiments, the pharmaceutical compositions are suitable for human administration. As used herein, the term “pharmaceutically acceptable” means approved by a regulatory agency of the federal or a state government or listed in the U.S. Pharmacopeia or other generally recognized pharmacopeia for use in animals, and more particularly in humans. The carrier can be a diluent, adjuvant, excipient, or vehicle with which the pharmaceutical composition is administered. Saline solutions and aqueous dextrose and glycerol solutions can also be employed as liquid carriers, including injectable solutions. Suitable excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. Examples of suitable pharmaceutical carriers are described in “Remington's Pharmaceutical Sciences” by E. W. Martin. In some embodiments, the pharmaceutical composition is sterilely formulated for administration into an individual. In some embodiments, the individual is a human. One of ordinary skilled in the art will appreciate that the formulation should suit the mode of administration.

In some embodiments, the pharmaceutical compositions of the present disclosure are formulated to be suitable for the intended route of administration to an individual. For example, the pharmaceutical composition may be formulated to be suitable for parenteral, intraperitoneal, colorectal, intraperitoneal, and intratumoral administration. In some embodiments, the pharmaceutical composition may be formulated for intravenous, oral, intraperitoneal, intratracheal, subcutaneous, intramuscular, topical, or intratumoral administration.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™. (BASF, Parsippany, N.J.), or phosphate buffered saline (PBS). In all cases, the composition should be sterile and should be fluid to the extent that easy syringability exists. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants, e.g., sodium dodecyl sulfate. Prevention of the action of microorganisms can be achieved by various antibacterial and antifungal agents, for example, parabens, chlorobutanol, phenol, ascorbic acid, thimerosal, and the like. In many cases, it will be generally to include isotonic agents, for example, sugars, polyalcohols such as mannitol, sorbitol, and/or sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above.

In some embodiments, the engineered immune cells of the disclosure can be formulated for administration to a subject using techniques known to the skilled artisan. For example, formulations comprising populations of engineered immune cells can include pharmaceutically acceptable excipient(s). Excipients included in the formulations will have different purposes depending, for example, on the engineered immune cells used and the mode of administration. Examples of generally used excipients included, without limitation: saline, buffered saline, dextrose, water-for-injection, glycerol, ethanol, and combinations thereof, stabilizing agents, solubilizing agents and surfactants, buffers and preservatives, tonicity agents, bulking agents, and lubricating agents. The formulations comprising engineered immune cells can have been prepared and cultured in the absence of non-human components, e.g., in the absence of animal serum. A formulation can include one population of engineered immune cells, or more than one, such as two, three, four, five, six or more populations of engineered immune cells.

Formulations comprising population(s) of engineered immune cells can be administered to a subject using modes and techniques known to the skilled artisan. Exemplary modes include, but are not limited to, intravenous injection. Other modes include, without limitation, intratumoral, intradermal, subcutaneous (S.C., s.q., sub-Q, Hypo), intramuscular (i.m.), intraperitoneal (i.p.), intra-arterial, intramedullary, intracardiac, intra-articular (joint), intrasynovial (joint fluid area), intracranial, intraspinal, and intrathecal (spinal fluids). Devices useful for parenteral injection of infusion of the formulations can be used to effect such administration.

### METHODS OF TREATMENT

Administration of any one of the therapeutic compositions described herein, e.g., engineered immune cells, nucleic acid molecules encoding glucose transporters, and pharmaceutical compositions, can be used to treat individuals in the treatment of relevant health conditions, such as proliferative diseases (e.g., cancers), autoimmune diseases, and microbial infections (e.g., viral infections). In some embodiments, one or more engineered immune cells, nucleic acid molecules, and pharmaceutical compositions as described herein can be incorporated into therapeutic agents for use in methods of treating a subject who has, who is suspected of having, or who may be at high risk for developing one or more health conditions, such as proliferative diseases (e.g., cancers), autoimmune diseases, and chronic infections. In some embodiments, the subject is a mammalian subject. In some embodiments, the subject has or is suspected of having a proliferative disease, an autoimmune disease, or an infection. In some embodiments, the proliferative disease is a cancer. In some embodiments, the subject is a patient under the care of a physician.

Accordingly, in one aspect, some embodiments of the disclosure relate to methods for preventing and/or treating a health condition in a subject in need thereof. In some embodiments, the methods include administering to the subject a composition of the disclosure. In some embodiments, the methods include administering to the subject a composition that includes an engineered immune cell of the disclosure. In some embodiments, the methods include administering to the subject a composition that includes a nucleic acid comprising a sequence encoding one or more glucose transporters. In some embodiments, the nucleic acid includes a sequence encoding one or more glucose transporters selected from the group consisting of GLUT1, GLUT2, GLUT3, GLUT4, GLUT6, GLUT8, GLUT9, GLUT10, GLUT11, and GLUT12. In some embodiments, the methods include administering to the subject a pharmaceutical composition as described herein.

In some embodiments, the methods include administering a therapeutically effective amount of a composition of the disclosure (e.g., engineered immune cells, nucleic acid molecules encoding glucose transporters, and pharmaceutical compositions) to a subject in need thereof. The term “effective amount”, “therapeutically effective amount”, or “pharmaceutically effective amount” of a subject engineered immune cell or pharmaceutical composition of the disclosure generally refers to an amount or number sufficient for a population of engineered immune cells or a pharmaceutical composition to accomplish a stated purpose relative to the absence of the engineered cell population or pharmaceutical composition (e.g., achieve the effect for which it is administered, treat a disease, reduce a signaling pathway, or reduce one or more symptoms of a disease or health condition). An example of an “effective amount” is an amount sufficient to contribute to the treatment, prevention, or reduction of a symptom or symptoms of a disease, which could also be referred to as a “therapeutically effective amount.” A “reduction” of a symptom means decreasing of the severity or frequency of the symptom(s), or elimination of the symptom(s). The exact amount of a T-cell population or composition including a “therapeutically effective amount” will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

Non-limiting exemplary embodiments of the treatment methods described herein can include one or more of the following features. In some embodiments, the health condition is a proliferative disease or an infection. Exemplary proliferative diseases can include, without limitation, angiogenic diseases, a metastatic diseases, tumorigenic diseases, neoplastic diseases and cancers. In some embodiments, the proliferative disease is a cancer. In some embodiments, the cancer is a pediatric cancer. In some embodiments, the cancer is a pancreatic cancer, a colon cancer, an ovarian cancer, a prostate cancer, a lung cancer, mesothelioma, a breast cancer, a urothelial cancer, a liver cancer, a head and neck cancer, a sarcoma, a cervical cancer, a stomach cancer, a gastric cancer, a melanoma, a uveal melanoma, a cholangiocarcinoma, multiple myeloma, leukemia, lymphoma, and glioblastoma.

In some embodiments, the cancer is a multiply drug resistant cancer or a recurrent cancer. It is contemplated that the compositions and methods disclosed here are suitable for both non-metastatic cancers and metastatic cancers. Accordingly, in some embodiments, the cancer is a non-metastatic cancer. In some other embodiments, the cancer is a metastatic cancer. In some embodiments, the composition administered to the subject inhibits metastasis of the cancer in the subject. In some embodiments, the administered composition inhibits tumor growth in the subject.

Exemplary proliferative diseases can include, without limitation, angiogenic diseases, a metastatic diseases, tumorigenic diseases, neoplastic diseases and cancers. In some embodiments, the proliferative disease is a cancer. The term “cancer” generally refers to a disease characterized by the rapid and uncontrolled growth of aberrant cells. The aberrant cells may form solid tumors or constitute a hematological malignancy. Cancer cells can spread locally or through the bloodstream and lymphatic system to other parts of the body. There are no specific limitations with respect to the cancers which can be treated by the compositions and methods of the present disclosure. Non-limiting examples of suitable cancers include ovarian cancer, renal cancer, breast cancer, prostate cancer, liver cancer, brain cancer, lymphoma, leukemia, cervical cancer, skin cancer, pancreatic cancer, colorectal cancer, lung cancer and the like.

Other cancers that can be suitable treated with the compositions and methods of the present disclosure include, but are not limited to, acute myeloblastic leukemia (AML), acute lymphoblastic leukemia (ALL), chronic myelocytic leukemia (CML), adrenal cortical cancer, anal cancer, aplastic anemia, bile duct cancer, bladder cancer, bone cancer, bone metastasis, brain cancers, central nervous system (CNS) cancers, peripheral nervous system (PNS) cancers, breast cancer, cervical cancer, colon and rectum cancer, endometrial cancer, esophagus cancer, Ewing's family of tumors (e.g. Ewing's sarcoma), eye cancer, transitional cell carcinoma, vaginal cancer, myeloproliferative disorders, nasal cavity and paranasal cancer, nasopharyngeal cancer, neuroblastoma, oral cavity and oropharyngeal cancer, osteosarcoma, ovarian cancer, pancreatic cancer, penile cancer, pituitary tumor, prostate cancer, retinoblastoma, gallbladder cancer, gastrointestinal carcinoid tumors, gastrointestinal stromal tumors, gestational trophoblastic disease, Non-Hodgkin's lymphoma, Hodgkin's lymphoma, childhood Non-Hodgkin's lymphoma, Kaposi's sarcoma, kidney cancer, laryngeal and hypopharyngeal cancer, liver cancer, lung cancer, lung carcinoid tumors, malignant mesothelioma, multiple myeloma, myelodysplastic syndrome, rhabdomyosarcoma, salivary gland cancer, sarcomas, melanoma skin cancer, non-melanoma skin cancers, stomach cancer, testicular cancer, thymus cancer, thyroid cancer, uterine cancer (e.g., uterine sarcoma), transitional cell carcinoma, vaginal cancer, vulvar cancer, mesothelioma, squamous cell or epidermoid carcinoma, bronchial adenoma, choriocarinoma, head and neck cancers, teratocarcinoma, or Waldenstrom's macroglobulinemia.

Particularly suitable cancers include, but are not limited to, breast cancer, ovarian cancer, lung cancer, pancreatic cancer, mesothelioma, leukemia, lymphoma, brain cancer, prostate cancer, multiple myeloma, melanoma, bladder cancer, bone sarcomas, soft tissue sarcomas, retinoblastoma, renal tumors, neuroblastoma, and carcinomas.

In some embodiments, the cancer is a multiply drug resistant cancer or a recurrent cancer. It is contemplated that the compositions and methods disclosed here are suitable for both non-metastatic cancers and metastatic cancers. Accordingly, in some embodiments, the cancer is a non-metastatic cancer. In some other embodiments, the cancer is a metastatic cancer. In some embodiments, the composition administered to the subject inhibits metastasis of the cancer in the subject. For example, in some embodiments, the composition administered to the subject can reduce metastatic nodules in the subject. In some embodiments, the administered composition inhibits tumor growth in the subject.

In some embodiments, the proliferative disease is an autoimmune disease. In some embodiments, the autoimmune disease is selected from the group consisting of rheumatoid arthritis, insulin-dependent diabetes mellitus, hemolytic anemias, rheumatic fever, thyroiditis, Crohn's disease, myasthenia gravis, glomerulonephritis, autoimmune hepatitis, multiple sclerosis, alopecia areata, psoriasis, vitiligo, dystrophic epidermolysis bullosa, systemic lupus erythematosus, moderate to severe plaque psoriasis, psoriatic arthritis, Crohn's disease, ulcerative colitis, and graft vs. host disease.

In some embodiments, the administered composition inhibits proliferation of a target cancer cell, and/or inhibits tumor growth of the cancer in the subject. For example, the target cell may be inhibited if its proliferation is reduced, if its pathologic or pathogenic behavior is reduced, if it is destroyed or killed, etc. Inhibition includes a reduction of the measured pathologic or pathogenic behavior of at least about 10%, about 15%, about 20%, about 25%, about 30%, about 35%, about 40%, about 45%, about 50%, about 55%, about 60%, about 65%, about 70%, about 75%, about 80%, about 85%, about 90%, or about 95%. In some embodiments, the methods include administering to the individual an effective number of the engineered immune cells disclosed herein, wherein the engineered immune cells inhibit the proliferation of the target cell and/or inhibit tumor growth of a target cancer in the subject compared to the proliferation of the target cell and/or tumor growth of the target cancer in subjects who have not been administered with the engineered immune cells.

Administration of the compositions described herein, e.g., engineered immune cells, nucleic acids, and pharmaceutical compositions, can be used in the stimulation of an immune response. In some embodiments, one or more of engineered immune cells, nucleic acids, and/or pharmaceutical compositions as described herein are administered to an individual after induction of remission of cancer with chemotherapy, or after autologous or allogeneic hematopoietic stem cell transplantation. In some embodiments, compositions described herein are administered to a subject in need of increasing the production of interferon gamma (IFNγ), tumor-necrosis factor alpha (TNFα), and/or interleukin-2 (IL-2) in the treated subject relative to the production of these molecules in subjects who have not been administered one of the therapeutic compositions disclosed herein.

In some embodiments, the administered composition confers an enhanced effector function of the immune cells. Examples of effector functions that are enhanced in the engineered immune cells include, but are not limited to growth rate (proliferation), death rate, death rate type, target cell inhibition (cytotoxicity), target cell killing, target cell survival, cluster of differentiation change, macrophage activation, B cell activation, cytokine production, in vivo persistence, and increased glycolytic flux. In some embodiments, an effector function of the immune cells including the composition of the disclosure is enhanced at levels that are at least 10% higher, such as at least 10% higher than about 10%, at least higher than about 20%, at least higher than about 30%, at least higher than about 40%, at least higher than about 50%, at least higher than about 60%, at least higher than about 70%, at least higher than about 80%, at least higher than about 90%, at least higher than about 2 times, higher than about three times, higher than about four time, higher than about five times, higher than about six times, higher than about seven times, higher than about eight times, higher than about nine times, higher than about 20 times, higher than about 50 times, higher than about 100 times, or higher than about 200 times compared to a reference immune cell. In some embodiments, the reference immune cell does not include a composition of the disclosure. In some embodiments, the administered composition confers an increased glycolytic flux in the immune cells. In some embodiments, the administered composition confers a glycolytic flux that is increased by at least 10%, such as at least about 10%, at least about 20%, at least about 30%, at least about 40%, at least about 50%, at least about 60%, at least about 70%, at least about 80%, at least about 90%, at least about 2 times, about three times, about four time, about five times, about six times, about seven times, about eight times, about nine times, about 20 times, about 50 times, about 100 times, or about 200 times compared to a reference immune cell.

An effective amount of the compositions described herein, e.g., engineered immune cells, nucleic acids, and/or pharmaceutical compositions, can be determined based on the intended goal, for example cancer regression. For example, where existing cancer is being treated, the amount of a composition disclosed herein to be administered may be greater than where administration of the composition is for prevention of cancer. One of ordinary skill in the art would be able to determine the amount of a composition to be administered and the frequency of administration in view of this disclosure. The quantity to be administered, both according to number of treatments and dose, also depends on the individual to be treated, the state of the individual, and the protection desired. Precise amounts of the composition also depend on the judgment of the practitioner and are peculiar to each subject. Frequency of administration could range from 1-2 days, to 2-6 hours, to 6-10 hours, to 1-2 weeks or longer depending on the judgment of the practitioner.

Determination of the amount of compositions to be administered will be made by one of skill in the art, and will in part be dependent on the extent and severity of cancer, and whether the engineered immune cells are being administered for treatment of existing cancer or prevention of cancer. For example, longer intervals between administration and lower amounts of compositions may be employed where the goal is prevention. For instance, amounts of compositions administered per dose may be 50% of the dose administered in treatment of active disease, and administration may be at weekly intervals. One of ordinary skill in the art, in light of this disclosure, would be able to determine an effective amount of compositions and frequency of administration. This determination would, in part, be dependent on the particular clinical circumstances that are present (e.g., type of cancer, severity of cancer).

In some embodiments, it may be desirable to provide a continuous supply of a composition disclosed herein to the subject to be treated, e.g., a patient. In some embodiments, continuous perfusion of the region of interest (such as a tumor) may be suitable. The time period for perfusion would be selected by the clinician for the particular subject and situation, but times could range from about 1-2 hours, to 2-6 hours, to about 6-10 hours, to about 10-24 hours, to about 1-2 days, to about 1-2 weeks or longer. Generally, the dose of the composition via continuous perfusion will be equivalent to that given by single or multiple injections, adjusted for the period of time over which the doses are administered.

In some embodiments, administration is by intravenous infusion. An effective amount of the engineered immune cells, nucleic acids, and/or pharmaceutical compositions disclosed herein can be determined based on the intended goal, for example tumor regression. For example, where existing cancer is being treated, the number of cells to be administered may be greater than where administration of the engineered immune cells disclosed herein is for prevention of cancer. One of ordinary skill in the art would be able to determine the number of cells to be administered and the frequency of administration in view of this disclosure. The quantity to be administered, both according to number of treatments and dose, also depends on the individual to be treated, the state of the individual, and the protection desired. Precise amounts of the therapeutic composition also depend on the judgment of the practitioner and are peculiar to each individual. Frequency of administration could range from 1-2 days, to 2-6 hours, to 6-10 hours, to 1-2 weeks or longer depending on the judgment of the practitioner. Generally, the dose of the therapeutic composition via continuous perfusion will be equivalent to that given by single or multiple injections, adjusted for the period of time over which the doses are administered.

### Administration of Engineered Immune Cells to a Subject

In some embodiments, the methods of the disclosure involve administering an effective amount or number of the engineered immune cells provided here to a subject in need thereof. This administering step can be accomplished using any method of implantation delivery in the art. For example, the engineered immune cells can be infused directly in the subject's bloodstream or otherwise administered to the subject.

In some embodiments, the methods disclosed herein include administering, which term is used interchangeably with the terms “introducing,” implanting,” and “transplanting,” engineered immune cells into an individual, by a method or route that results in at least partial localization of the introduced cells at a desired site such that a desired effect(s) is/are produced. The engineered immune cells or their differentiated progeny can be administered by any appropriate route that results in delivery to a desired location in the individual where at least a portion of the administered cells or components of the cells remain viable. The period of viability of the cells after administration to a subject can be as short as a few hours, e.g., twenty-four hours, to a few days, to as long as several years, or even the lifetime of the individual, e.g., long-term engraftment.

When provided prophylactically, the engineered immune cells described herein can be administered to a subject in advance of any symptom of a disease or health condition to be treated. Accordingly, in some embodiments the prophylactic administration of an engineered immune cell population prevents the occurrence of symptoms of the disease or health condition.

When provided therapeutically in some embodiments, engineered immune cells are provided at (or after) the onset of a symptom or indication of a disease or health condition, e.g., upon the onset of disease or health condition.

For use in the various embodiments described herein, an effective amount of engineered immune cells as disclosed herein, can be at least 10² cells, at least 5×10² cells, at least 10³ cells, at least 5×10³ cells, at least 10⁴ cells, at least 5×10⁴ cells, at least 10⁵ cells, at least 2×10⁵ cells, at least 3×10⁵ cells, at least 4×10⁵ cells, at least 5×10⁵ cells, at least 6×10⁵ cells, at least 7×10⁵ cells, at least 8×10⁵ cells, at least 9×10⁵ cells, at least 1×10⁶ cells, at least 2×10⁶ cells, at least 3×10⁶ cells, at least 4×10⁶ cells, at least 5×10⁶ cells, at least 6×10⁶ cells, at least 7×10⁶ cells, at least 8×10⁶ cells, at least 9×10⁶ cells, or multiples thereof.

In some embodiments, the engineered immune cells are non-autologous to the subject in need of treatment. In some embodiments, the adoptive cell therapy is an allogeneic adoptive cell therapy. For example, in some embodiments, the engineered immune cells are allogeneic to the subject in need of treatment. In an allogeneic adoptive cell therapy, the engineered immune cells are not derived from the individual receiving the adoptive cell therapy. Allogeneic cell therapy generally refers to a therapy whereby the individual (donor) who provides the immune cells is a different individual (of the same species) than the individual receiving the cell therapy. For example, a population of engineered immune cells being administered to an individual is derived from one more unrelated donors, or from one or more non-identical siblings. Accordingly, the engineered immune cells can be derived from one or more donors or can be obtained from an autologous source. In some embodiments, the engineered immune cells are expanded in culture prior to administration to a subject in need thereof.

In some embodiments, the delivery of a cell composition (e.g., a composition including a plurality of engineered immune cells according to any of the cells described herein) into a subject by a method or route results in at least partial localization of the cell composition at a desired site. A composition including engineered immune cells can be administered by any appropriate route that results in effective treatment in the subject, e.g., administration results in delivery to a desired location in the subject where at least a portion of the composition delivered, e.g., at least 1×10⁴ cells, is delivered to the desired site for a period of time. Exemplary modes of suitable administration include injection, infusion, and instillation. “Injection” includes, without limitation, intravenous, intramuscular, intra-arterial, intrathecal, intraventricular, intracapsular, intraorbital, intracardiac, intradermal, intraperitoneal, transtracheal, subcutaneous, subcuticular, intraarticular, subcapsular, subarachnoid, intraspinal, intracerebrospinal, and intrasternal injection and infusion. In some embodiments, the route is intravenous. For the delivery of cells, delivery by injection or infusion is often considered a standard mode of administration.

In some embodiments, the engineered immune cells are administered systemically, e.g., via infusion or injection. For example, a population of engineered immune cells as described herein are administered other than directly into a target site, tissue, or organ, such that it enters, the subject's circulatory system and, thus, is subject to metabolism and other similar biological processes.

The efficacy of a treatment including any of the compositions provided herein for the prevention or treatment of a disease or health condition can be determined by a skilled clinician. However, one skilled in the art will appreciate that a prevention or treatment is considered effective if any one or all of the signs or symptoms or markers of disease are improved or ameliorated. Efficacy can also be measured by failure of a subject to worsen as assessed by decreased hospitalization or need for medical interventions (e.g., progression of the disease is halted or at least slowed). Methods of measuring these indicators are known to those of skill in the art and/or described herein. Treatment includes any treatment of a disease in a subject or an animal (some non-limiting examples include a human, or a mammal) and includes: (1) inhibiting the disease, e.g., arresting, or slowing the progression of symptoms; or (2) relieving the disease, e.g., causing regression of symptoms; and (3) preventing or reducing the likelihood of the development of symptoms.

Measurement of the degree of efficacy is based on parameters selected with regard to the disease being treated and the symptoms experienced. In general, a parameter is selected that is known or accepted as correlating with the degree or severity of the disease, such as a parameter accepted or used in the medical community. For example, in the treatment of a solid cancer, suitable parameters can include reduction in the number and/or size of metastases, number of months of progression-free survival, overall survival, stage or grade of the disease, the rate of disease progression, the reduction in diagnostic biomarkers (for example without limitation, a reduction in circulating tumor DNA or RNA, a reduction in circulating cell-free tumor DNA or RNA, and the like), and combinations thereof. It will be understood that the effective dose and the degree of efficacy will generally be determined with relation to a single subject and/or a group or population of subjects. Therapeutic methods of the disclosure reduce symptoms and/or disease severity and/or disease biomarkers by at least about 1, 2, 3, 4, 5, 10, 15, 20, 25, 30, 35, 40, 45, 50, 55, 60, 65, 70, 75, 80, 85, 90, 95, 96, 97, 98, 99,or 100%.

As discussed above, a therapeutically effective amount of a pharmaceutical composition can be an amount of the pharmaceutical composition that is sufficient to promote a particular beneficial effect when administered to a subject, such as one who has, is suspected of having, or is at risk for a disease or health condition. In some embodiments, an effective amount includes an amount sufficient to prevent or delay the development of a symptom of the disease or health condition, alter the course of a symptom of the disease or health condition (for example but not limited to, slow the progression of a symptom of the disease), or reverse a symptom of the disease or health condition. It is understood that for any given case, an appropriate effective amount can be determined by one of ordinary skill in the art using routine experimentation.

### Additional Therapies

As discussed above, any one of the compositions as disclosed herein, e.g., engineered immune cells and pharmaceutical compositions, can be administered to a subject in need thereof as a single therapy (e.g., monotherapy). In addition or alternatively, in some embodiments of the disclosure, one or more of the engineered immune cells and pharmaceutical compositions described herein can be administered to the subject in combination with one or more additional (e.g., supplementary) therapies, e.g., at least one, two, three, four, or five additional therapies. Suitable therapies to be administered in combination with the compositions of the disclosure include, but are not limited to chemotherapy, radiotherapy, immunotherapy, hormonal therapy, toxin therapy, targeted therapy, and surgery. Other suitable therapies include therapeutic agents such as chemotherapeutics, anti-cancer agents, and anti-cancer therapies.

Administration “in combination with” one or more additional therapies includes simultaneous (concurrent) and consecutive administration in any order. In some embodiments, the one or more additional therapies is selected from the group consisting of chemotherapy, radiotherapy, immunotherapy, hormonal therapy, toxin therapy, and surgery. The term chemotherapy as used herein encompasses anti-cancer agents. Various classes of anti-cancer agents can be suitably used for the methods disclosed herein. Non-limiting examples of anti-cancer agents include: alkylating agents, antimetabolites, anthracyclines, plant alkaloids, topoisomerase inhibitors, podophyllotoxin, antibodies (e.g., monoclonal or polyclonal), tyrosine kinase inhibitors (e.g., imatinib mesylate (Gleevec® or Glivec®)), hormone treatments, soluble receptors and other antineoplastics.

Topoisomerase inhibitors are also another class of anti-cancer agents that can be used herein. Topoisomerases are essential enzymes that maintain the topology of DNA. Inhibition of type I or type II topoisomerases interferes with both transcription and replication of DNA by upsetting proper DNA supercoiling. Some type I topoisomerase inhibitors include camptothecins such as irinotecan and topotecan. Examples of type II inhibitors include amsacrine, etoposide, etoposide phosphate, and teniposide. These are semisynthetic derivatives of epipodophyllotoxins, alkaloids naturally occurring in the root of American Mayapple (Podophyllum peltatum).

Antineoplastics include the immunosuppressant dactinomycin, doxorubicin, epirubicin, bleomycin, mechlorethamine, cyclophosphamide, chlorambucil, ifosfamide. The antineoplastic compounds generally work by chemically modifying a cell's DNA.

Alkylating agents can alkylate many nucleophilic functional groups under conditions present in cells. Cisplatin and carboplatin, and oxaliplatin are alkylating agents. They impair cell function by forming covalent bonds with the amino, carboxyl, sulfhydryl, and phosphate groups in biologically important molecules.

Vinca alkaloids bind to specific sites on tubulin, inhibiting the assembly of tubulin into microtubules (M phase of the cell cycle). The vinca alkaloids include: vincristine, vinblastine, vinorelbine, and vindesine.

Anti-metabolites resemble purines (azathioprine, mercaptopurine) or pyrimidine and prevent these substances from becoming incorporated in to DNA during the “S” phase of the cell cycle, stopping normal development and division. Anti-metabolites also affect RNA synthesis.

Plant alkaloids and terpenoids are obtained from plants and block cell division by preventing microtubule function. Since microtubules are vital for cell division, without them, cell division cannot occur. The main examples are vinca alkaloids and taxanes.

Podophyllotoxin is a plant-derived compound which has been reported to help with digestion as well as used to produce two other cytostatic drugs, etoposide and teniposide. They prevent the cell from entering the G1 phase (the start of DNA replication) and the replication of DNA (the S phase).

Taxanes as a group includes paclitaxel and docetaxel. Paclitaxel is a natural product, originally known as Taxol and first derived from the bark of the Pacific Yew tree. Docetaxel is a semi-synthetic analogue of paclitaxel. Taxanes enhance stability of microtubules, preventing the separation of chromosomes during anaphase.

In some embodiments, the anti-cancer agents can be selected from remicade, docetaxel, celecoxib, melphalan, dexamethasone (Decadron®), steroids, gemcitabine, cisplatinum, temozolomide, etoposide, cyclophosphamide, temodar, carboplatin, procarbazine, gliadel, tamoxifen, topotecan, methotrexate, gefitinib (Iressa®), taxol, taxotere, fluorouracil, leucovorin, irinotecan, xeloda, CPT-11, interferon alpha, pegylated interferon alpha (e.g., PEG INTRON-A), capecitabine, cisplatin, thiotepa, fludarabine, carboplatin, liposomal daunorubicin, cytarabine, doxetaxol, pacilitaxel, vinblastine, IL-2, GM-CSF, dacarbazine, vinorelbine, zoledronic acid, palmitronate, biaxin, busulphan, prednisone, bortezomib (Velcade®), bisphosphonate, arsenic trioxide, vincristine, doxorubicin (Doxil®), paclitaxel, ganciclovir, adriamycin, estrainustine sodium phosphate (Emcyt®), sulindac, etoposide, and combinations of any thereof.

In other embodiments, the anti-cancer agent can be selected from bortezomib, cyclophosphamide, dexamethasone, doxorubicin, interferon-alpha, lenalidomide, melphalan, pegylated interferon-alpha, prednisone, thalidomide, or vincristine.

In some embodiments, the methods of prevention and/or treatment as described herein further include an immunotherapy. In some embodiments, the immunotherapy includes administration of one or more checkpoint inhibitors. Accordingly, some embodiments of the methods of treatment described herein include further administration of a compound that inhibits one or more immune checkpoint molecules. Non-limiting examples of immune checkpoint molecules include CTLA4, PD-1, PD-L1, A2AR, B7-H3, B7-H4, TIM3, and combinations of any thereof. In some embodiments, the compound that inhibits the one or more immune checkpoint molecules includes an antagonistic antibody. Examples of antagonistic antibodies suitable for the compositions and methods disclosed herein include, but are not limited to, ipilimumab, nivolumab, pembrolizumab, durvalumab, atezolizumab, tremelimumab, and avelumab.

In some aspects, the one or more anti-cancer therapy is radiation therapy. In some embodiments, the radiation therapy can include the administration of radiation to kill cancerous cells. Radiation interacts with molecules in the cell such as DNA to induce cell death. Radiation can also damage the cellular and nuclear membranes and other organelles. Depending on the radiation type, the mechanism of DNA damage may vary as does the relative biologic effectiveness. For example, heavy particles (i.e. protons, neutrons) damage DNA directly and have a greater relative biologic effectiveness. Electromagnetic radiation results in indirect ionization acting through short-lived, hydroxyl free radicals produced primarily by the ionization of cellular water. Clinical applications of radiation consist of external beam radiation (from an outside source) and brachytherapy (using a source of radiation implanted or inserted into the patient). External beam radiation consists of X-rays and/or gamma rays, while brachytherapy employs radioactive nuclei that decay and emit alpha particles, or beta particles along with a gamma ray. Radiation also contemplated herein includes, for example, the directed delivery of radioisotopes to cancer cells. Other forms of DNA damaging factors are also contemplated herein such as microwaves and UV irradiation.

Radiation may be given in a single dose or in a series of small doses in a dose-fractionated schedule. The amount of radiation contemplated herein ranges from about 1 to about 100 Gy, including, for example, about 5 to about 80, about 10 to about 50 Gy, or about 10 Gy. The total dose may be applied in a fractioned regime. For example, the regime may include fractionated individual doses of 2 Gy. Dosage ranges for radioisotopes vary widely, and depends on the half-life of the isotope and the strength and type of radiation emitted. When the radiation includes use of radioactive isotopes, the isotope may be conjugated to a targeting agent, such as a therapeutic antibody, which carries the radionucleotide to the target tissue (e.g., tumor tissue).

Surgery described herein includes resection in which all or part of a cancerous tissue is physically removed, exercised, and/or destroyed. Tumor resection refers to physical removal of at least part of a tumor. In addition to tumor resection, treatment by surgery includes laser surgery, cryosurgery, electrosurgery, and microscopically controlled surgery (Mohs surgery). Removal of pre-cancers or normal tissues is also contemplated herein.

Accordingly, in some embodiments, the methods of the disclosure include administration of a composition disclosed herein to a subject individually as a single therapy (e.g., monotherapy). In some embodiments, a composition of the disclosure is administered to a subject as a first therapy in combination with a second therapy. In some embodiments, the second therapy is selected from the group consisting of chemotherapy, radiotherapy, immunotherapy, hormonal therapy, toxin therapy, and surgery. In some embodiments, the first therapy and the second therapy are administered concomitantly. In some embodiments, the first therapy is administered at the same time as the second therapy. In some embodiments, the first therapy and the second therapy are administered sequentially. In some embodiments, the first therapy is administered before the second therapy. In some embodiments, the first therapy is administered after the second therapy. In some embodiments, the first therapy is administered before and/or after the second therapy. In some embodiments, the first therapy and the second therapy are administered in rotation. In some embodiments, the first therapy and the second therapy are administered together in a single formulation.

### KITS

Also provided herein are kits for the practice of a method described herein. A kit can include one or more of the engineered immune cells, nucleic acids, and/or pharmaceutical compositions as described and provided herein. For examples, provided herein, in some embodiments, are kits that include one or more engineered immune cells of the disclosure. In some embodiments, provided herein are kits that include one or more pharmaceutical compositions of the disclosure. In some embodiments, the kits of disclosure further include written instructions for making the engineered immune cells, nucleic acids, and/or pharmaceutical compositions of the disclosure and using the same.

In some embodiments, the kits of the disclosure further include one or more syringes (including pre-filled syringes) and/or catheters (including pre-filled syringes) used to administer one any of the provided immune cells, nucleic acids, and pharmaceutical compositions to a subject in need thereof. In some embodiments, a kit can have one or more additional therapeutic agents that can be administered simultaneously or sequentially with the other kit components for a desired purpose, e.g., for modulating an activity of a cell, inhibiting a target cancer cell, or treating a health condition in a subject in need thereof.

For example, any of the above-described kits can further include one or more additional reagents, where such additional reagents can be selected from: dilution buffers; reconstitution solutions, wash buffers, control reagents, control expression vectors, negative control T-cell populations, positive control T-cell populations, reagents for ex vivo production of the T-cell populations.

In some embodiments, the components of a kit can be in separate containers. In some other embodiments, the components of a kit can be combined in a single container. For example, in some embodiments of the disclosure, the kit includes one or more of the provided immune cells, nucleic acids, and/or pharmaceutical compositions as described herein in one container (e.g., in a sterile glass or plastic vial) and a further therapeutic agent in another container (e.g., in a sterile glass or plastic vial).

In some embodiments, a kit can further include instructions for using the components of the kit to practice the methods disclosed herein. For example, the kit can include a package insert including information concerning the pharmaceutical compositions and dosage forms in the kit. Generally, such information aids patients and physicians in using the enclosed pharmaceutical compositions and dosage forms effectively and safely. For example, the following information regarding a combination of the disclosure may be supplied in the insert: pharmacokinetics, pharmacodynamics, clinical studies, efficacy parameters, indications and usage, contraindications, warnings, precautions, adverse reactions, overdosage, proper dosage and administration, how supplied, proper storage conditions, references, manufacturer/distributor information and intellectual property information.

In some embodiments, a kit can include further instructions for using the components of the kit to practice the methods disclosed herein. The instructions for practicing the methods are generally recorded on a suitable recording medium. For example, the instructions can be printed on a substrate, such as paper or plastic, etc. The instructions can be present in the kit as a package insert, in the labeling of the container of the kit or components thereof (e.g., associated with the packaging or sub-packaging), etc. The instructions can be present as an electronic storage data file present on a suitable computer readable storage medium, e.g. CD-ROM, diskette, flash drive, etc. In some instances, the actual instructions are not present in the kit, but means for obtaining the instructions from a remote source (e.g., via the internet), can be provided. An example of this embodiment is a kit that includes a web address where the instructions can be viewed and/or from which the instructions can be downloaded. As with the instructions, this means for obtaining the instructions can be recorded on a suitable substrate.

Each of the aspects and embodiments described herein are capable of being used together, unless excluded either explicitly or clearly from the context of the embodiment or aspect.

All publications and patent applications mentioned in this disclosure are herein incorporated by reference to the same extent as if each individual publication or patent application was specifically and individually indicated to be incorporated by reference.

No admission is made that any reference cited herein constitutes prior art. The discussion of the references states what their authors assert, and the Applicant reserves the right to challenge the accuracy and pertinence of the cited documents. It will be clearly understood that, although a number of information sources, including scientific journal articles, patent documents, and textbooks, are referred to herein; this reference does not constitute an admission that any of these documents forms part of the common general knowledge in the art.

The discussion of the general methods given herein is intended for illustrative purposes only. Other alternative methods and alternatives will be apparent to those of skill in the art upon review of this disclosure, and are to be included within the spirit and purview of this application.

### EXAMPLES

The practice of the present invention will employ, unless otherwise indicated, conventional techniques of molecular biology, microbiology, cell biology, biochemistry, nucleic acid chemistry, and immunology, which are well known to those skilled in the art. Such techniques are explained fully in the literature, such as Sambrook, J., & Russell, D. W. (2012). Molecular Cloning: A Laboratory Manual (4th ed.). Cold Spring Harbor, N.Y.: Cold Spring Harbor Laboratory and Sambrook, J., & Russel, D. W. (2001). Molecular Cloning: A Laboratory Manual (3rd ed.). Cold Spring Harbor, N.Y.: Cold Spring Harbor Laboratory (jointly referred to herein as “Sambrook”); Ausubel, F. M. (1987). Current Protocols in Molecular Biology. New York, N.Y.: Wiley (including supplements through 2014); Bollag, D. M. et al. (1996). Protein Methods. New York, N.Y.: Wiley-Liss; Huang, L. et al. (2005). Nonviral Vectors: Gene Therapy and Neuroscience Applications. San Diego, Calif.: Academic Press; Lefkovits, I. (1997). The Immunology Methods Manual: The Comprehensive Sourcebook of Techniques. San Diego, Calif.: Academic Press; Doyle, A. et al. (1998). Cell and Tissue Culture: Laboratory Procedures in Biotechnology. New York, N.Y.: Wiley; Mullis, K. B., Ferré, F. & Gibbs, R. (1994). PCR: The Polymerase Chain Reaction. Boston: Birkhauser Publisher; Greenfield, E. A. (2014). Antibodies: A Laboratory Manual (2nd ed.). New York, N.Y.: Cold Spring Harbor Laboratory Press; Beaucage, S. L. et al. (2000). Current Protocols in Nucleic Acid Chemistry. New York, N.Y.: Wiley, (including supplements through 2014); and Makrides, S. C. (2003). Gene Transfer and Expression in Mammalian Cells. Amsterdam, NL: Elsevier Sciences B.V., the disclosures of which are incorporated herein by reference.

Additional embodiments are disclosed in further detail in the following examples, which are provided by way of illustration and are not in any way intended to limit the scope of this disclosure or the claims.

### Example 1

### Overexpression of GLUT1 Increases Cytokine Secretion of Exhausted and Non-Exhausted CAR T Cells Activated through their TCR or their CAR

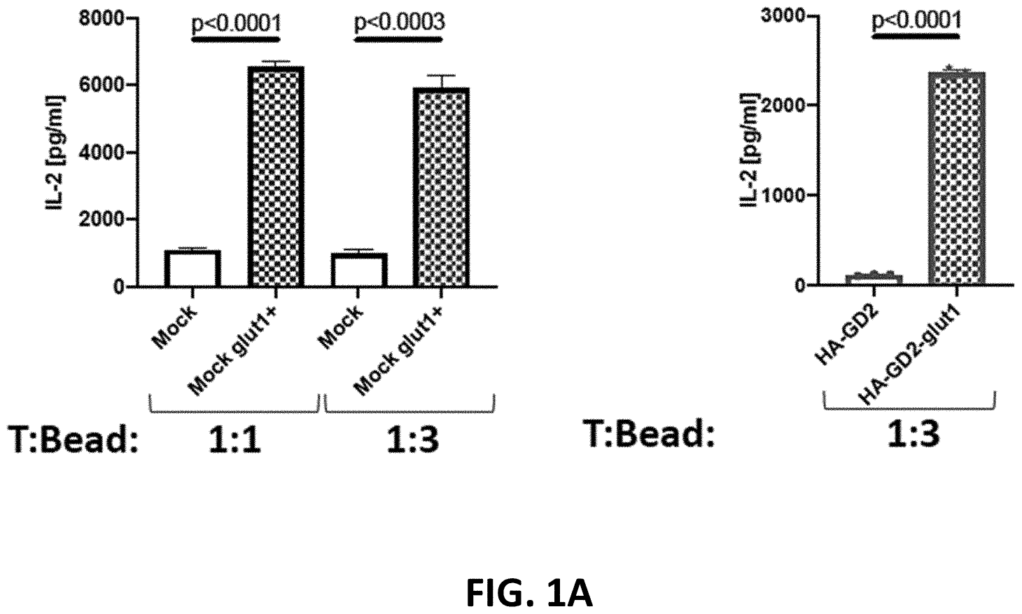

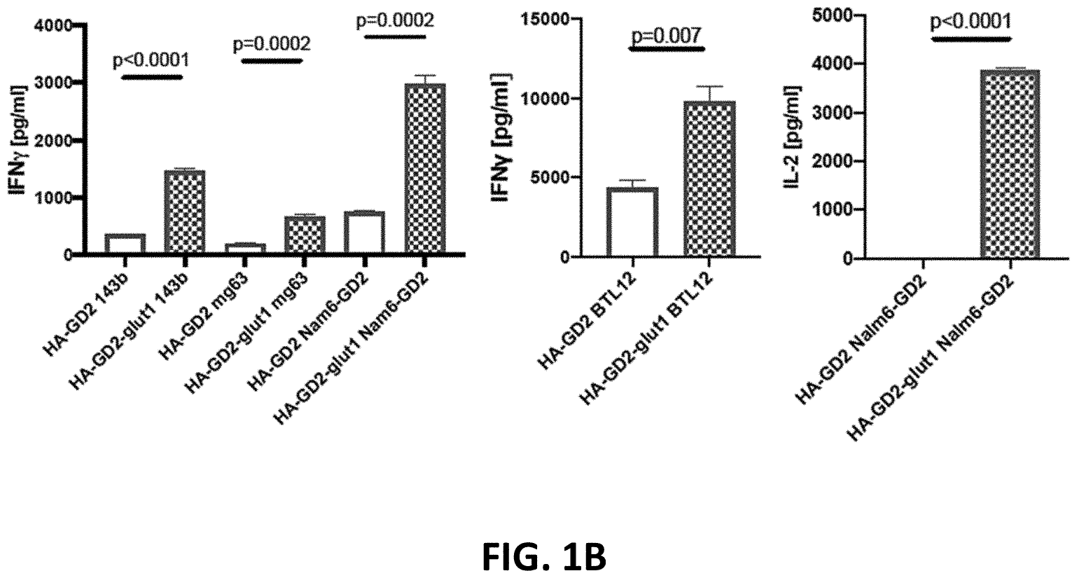

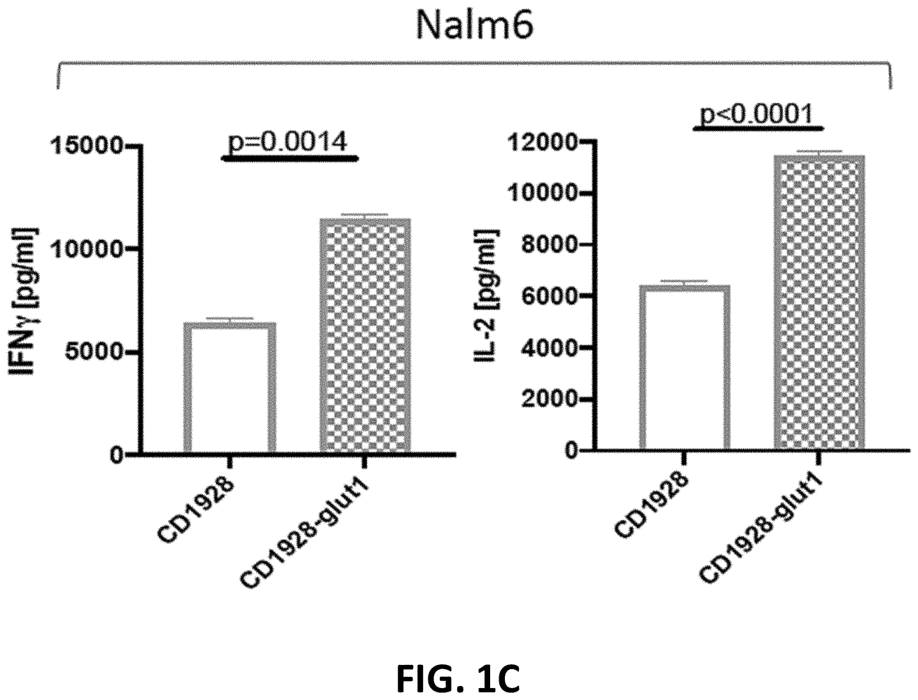

This Example describes the results of experiments performed to illustrate that overexpression of GLUT1 increases cytokine secretion of exhausted CAR T cells and non-exhausted CAR T cells. In these experiments, enriched CD3 human T cells were transduced using a lentiviral plasmid expressing GFP-GLUT1 cDNA. It was observed that GLUT1overexpression did not affect cell viability but resulted in increased production of IL-2 upon stimulation using anti-CD3/anti-CD28 beads at low (1:1) T cell:bead ratio and high T cell:bead ratio (1:3) as compared with non-transduced cells (see, e.g., FIG. 1A, left panel). Additional experiments were performed to demonstrate that T cells that demonstrate hallmark features of exhaustion as a result of expression of a tonic signaling (HA) GD2 CAR, also demonstrated increased production of IL-2 and IFNyγ when stimulated through the TCR with anti-CD3/anti-CD28 beads (1:3) (see, e.g., FIG. 1A, right panel) or when stimulated specifically through the CAR using an CAR-specific antigen expressing solid tumor line or leukemic tumor line (see, e.g., FIG. 1B).

In addition, CD1928 CAR T cells +/−GLUT1 were generated and stimulated through the CAR with tumor lines expressing CD19 antigen as a model for non-exhausted cells. It was observed that a significant increase in the amount of IL-2 and IFNγ secreted by healthy population of CAR T cells was detected when GLUT1 was overexpressed (see, e.g., FIG. 1C).

### Example 2

### GLUT1 Overexpression Increases Proliferation and Expansion of Exhausted CAR T Cells

This Example describes the results of experiments performed to demonstrate that GLUT1 overexpression increases proliferation and expansion of exhausted CAR T cells.

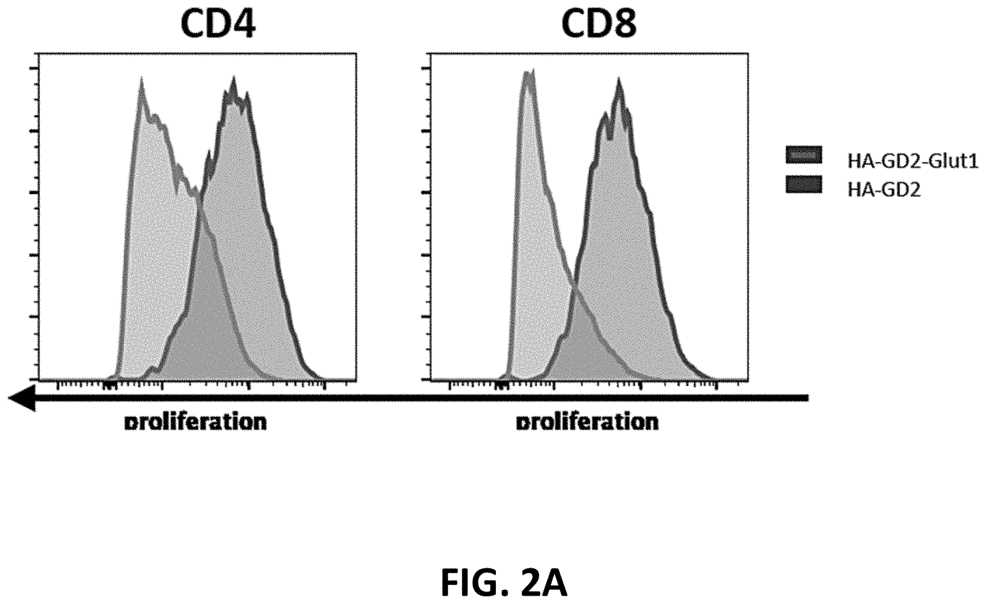

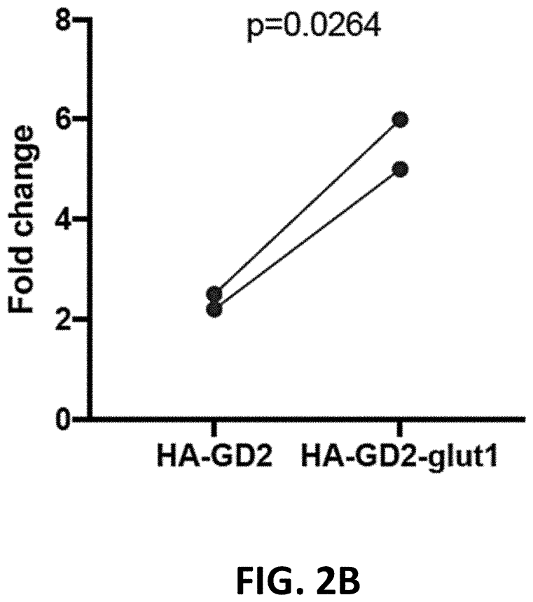

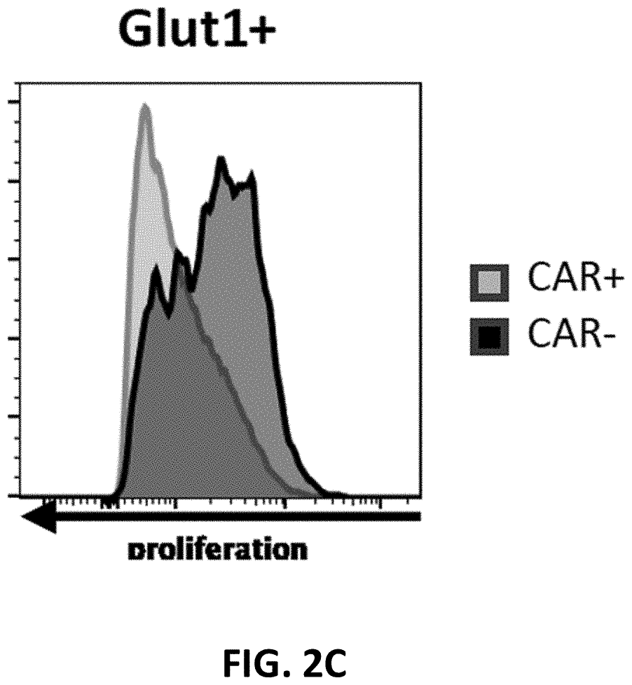

Cell proliferation is an energy consuming process. Therefore, it was hypothesized that overexpressing GLUT1 would enhance CAR T cell proliferation, and that this effect would be exaggerated in the context of CAR T cell exhaustion, where proliferation is suboptimal. It was observed that overexpression of GLUT1 on the surface of exhausted tonic signaling GD2 CAR T cells was associated with increased proliferative rate and expansion (see, e.g., FIGS. 2A-2B). Of note, the increased cell proliferation was dependent upon a signal derived from the tonic signaling CAR, as GLUT1⁺ GD2 CAR⁺ T cell proliferated at a substantially greater rate than GLUT1⁺ CAR⁻ cells (see, e.g., FIG. 2C), providing evidence that GLUT1 overexpression does not induce antigen independent proliferation.

### Example 3

### Constitutively High Expression of GLUT1 Improves CAR T Cell Function Against Low Antigen Density Tumor Cell Lines In Vitro

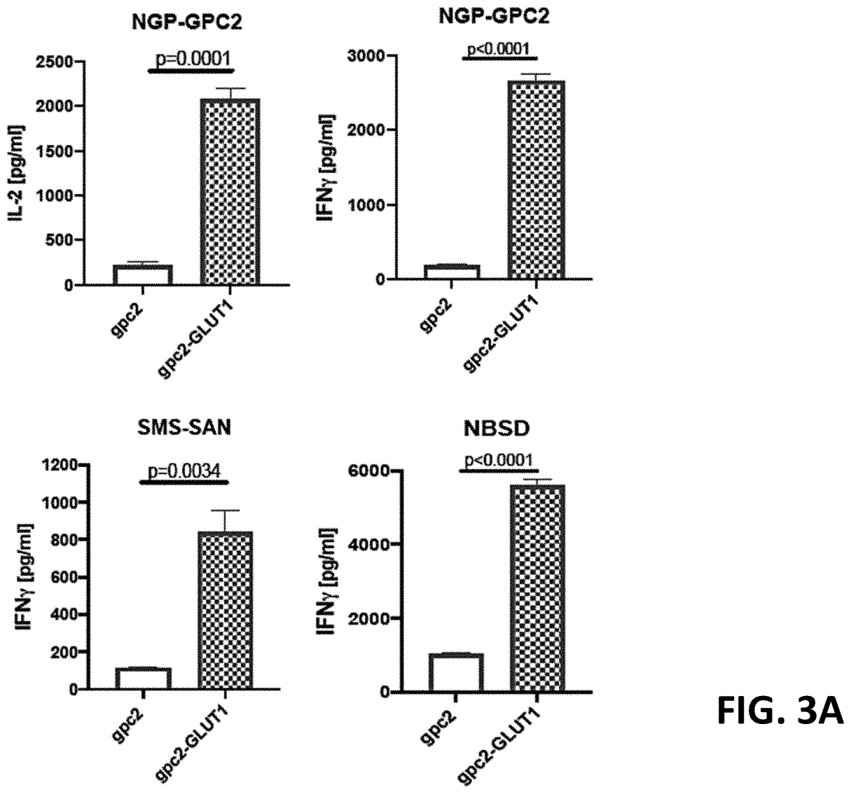

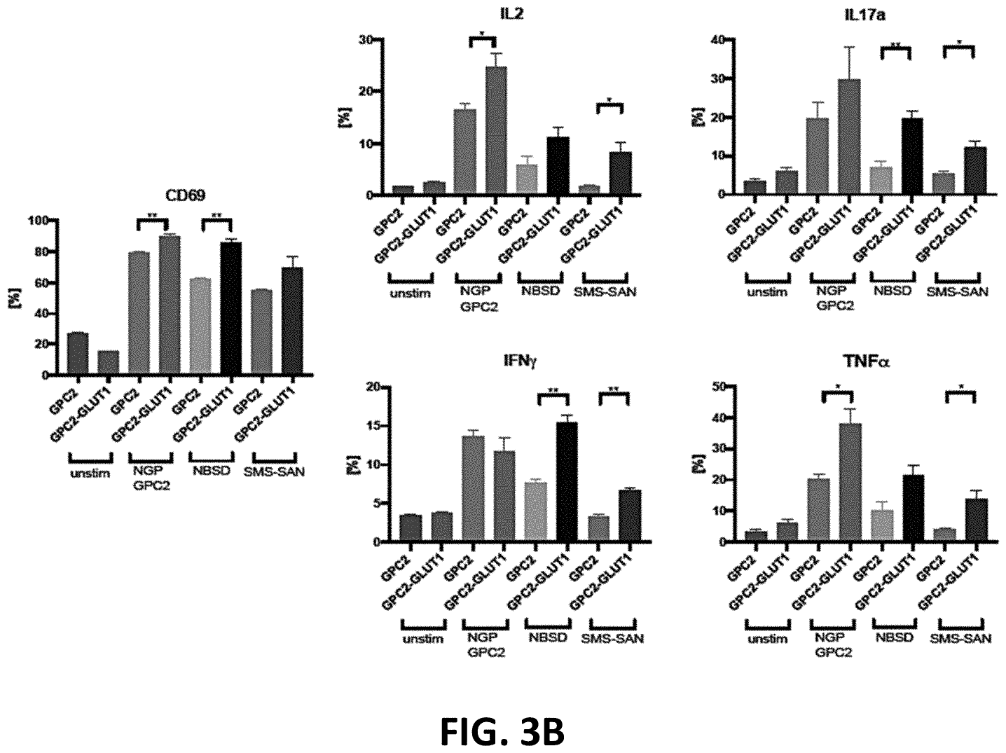

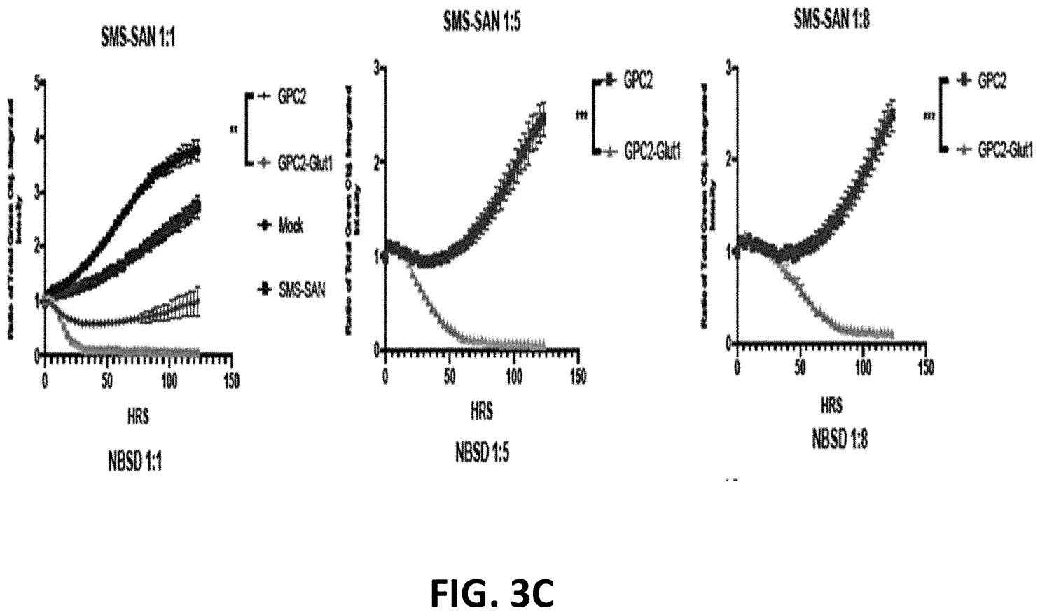

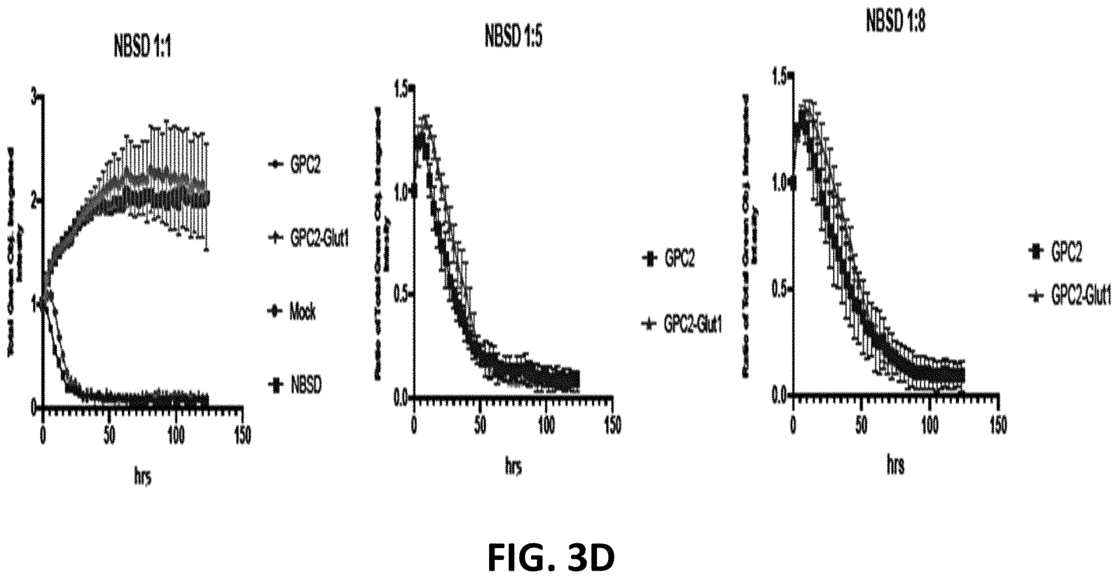

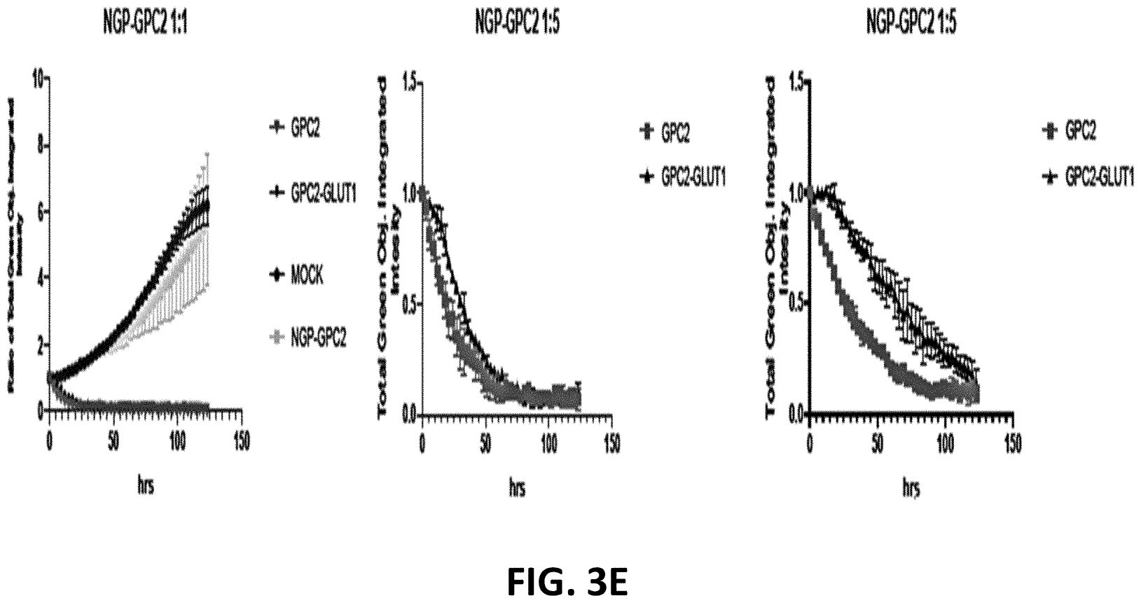

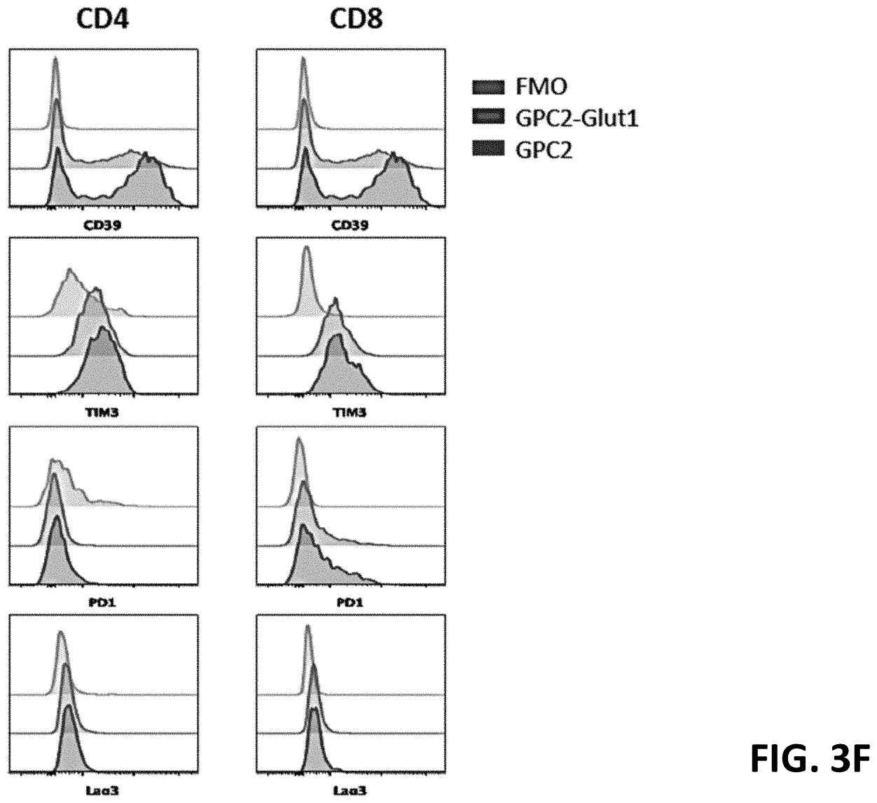

This Example describes the results of experiments performed to demonstrate that constitutively high expression of GLUT1 improves CAR T cell function against low antigen density tumor cell lines in vitro. In these experiments, GPC2 and GPC2-GLUT1 CAR T cells were co-incubated with tumor cell lines expressing different amounts of Glycipan antigen (GPC2) on the surface. NGP-GPC2 was an isogenic tumor line that expressed GPC2 at a level 5×higher than native expression of that molecule on NBSD line or SMS-SAN line. Analyses of co-culture supernatant showed that when compared with GPC2-CAR T cells, GPC2-GLUT1 CAR T cells secreted significantly more IL-2 and IFNγ when stimulated with NGP-GPC2 (high GPC2). It was also observed that GPC2-GLUT1 CAR T cells secreted significantly more IFNy in response to both NBSD and SMS-SAN (see, e.g., FIG. 3A).

In addition, a higher percentage of CD69⁺ cells was observed when activated with NGP-GPC2 and NBSD and increased cytokine production at the single cell level in response to all three tumor lines (see, e.g., FIG. 3B). It was also observed that both constructs eradicated NGP-GPC2 and NBSD when challenged with 1:8 E:T ratios but only GPC2-GLUT1 CAR T cells killed SMS-SAN tumor even at very low 1:8 E:T ratio (see, e.g., FIGS. 3C-3E).

Furthermore, when the expression of exhaustion markers on the surface of GPC2-CAR T cells +/+GLUT1 was compared, higher levels of expression of CD39, TIM3, PD1 or Lag3 in cells overexpressing GLUT1 were not observed (see, e.g., FIG. 3F).

### Example 4

### GPC2-GLUT1 CAR T Cells have Higher Proliferative Potential

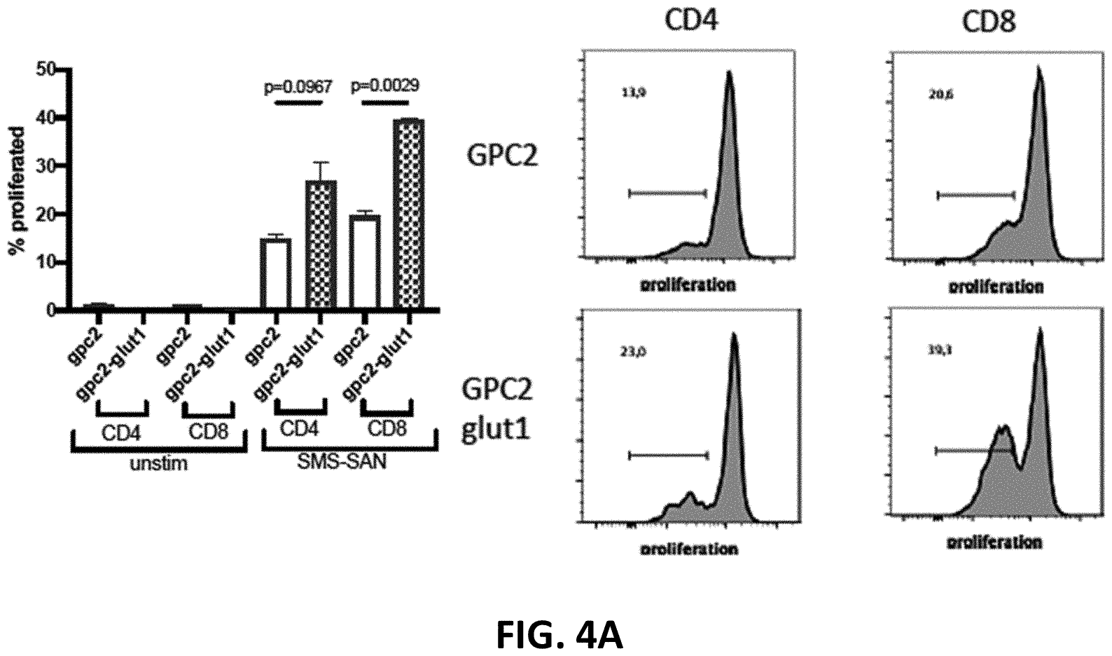

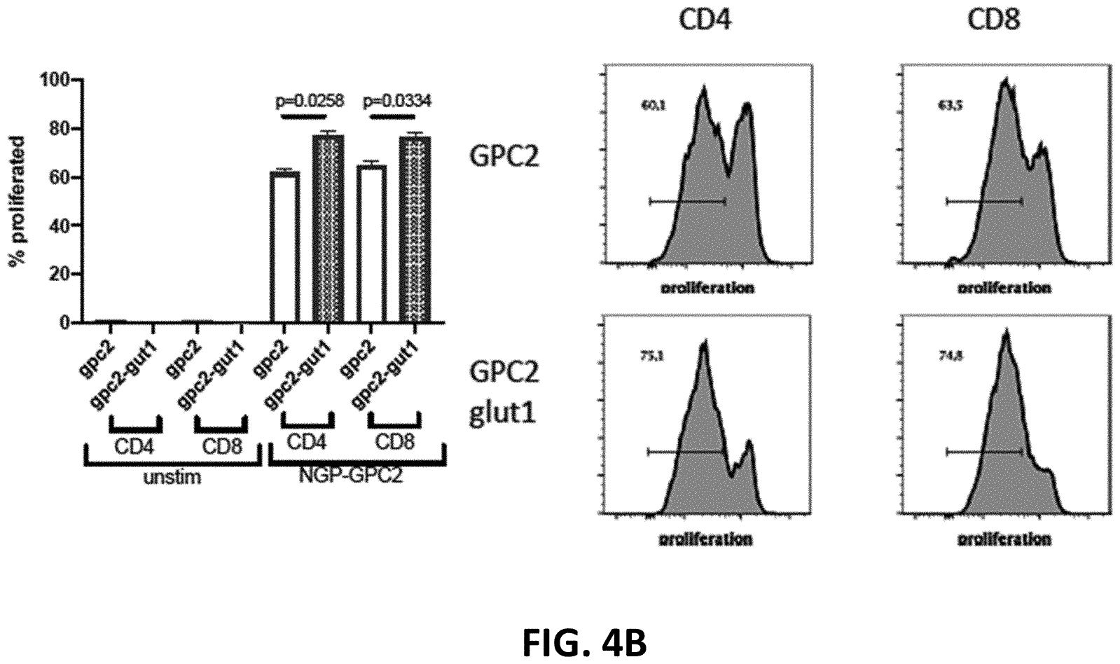

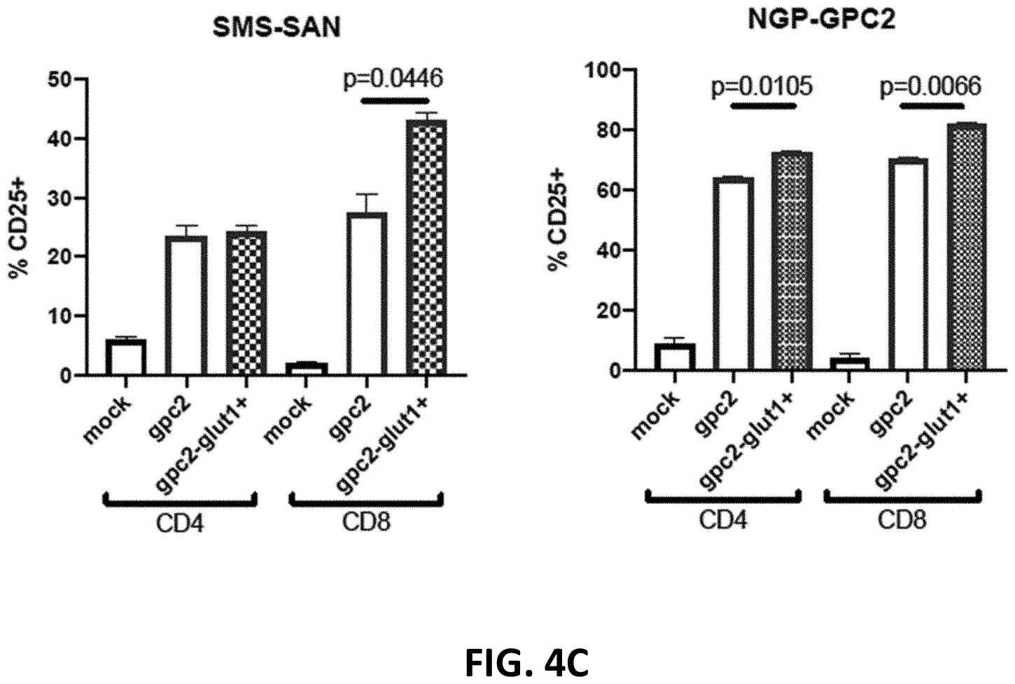

This Example describes the results of experiments performed to illustrate that GPC2-GLUT1 CAR T cells have higher proliferative potential. In these experiments, the effect of GLUT1 overexpression was tested on healthy GPC2-CAR T cells. It was observed that GLUT1 overexpression had no effect on the proliferation of non-stimulated GPC2 CAR T cells. On the other hand, when CAR T cells were stimulated through their CAR with SMS-SAN or NGP-GPC2 tumor lines, both CD4 and CD8 GPC2-GLUT1 CAR T cells proliferated at higher rates, as compare to GPC2 CAR T cells (see, e.g., FIGS. 4A-4B). Importantly, increased proliferation was found to be associated with more CD25⁺ activated cells (see, e.g., FIG. 4C).

### Example 5

### Co-Expression of GLUT1 Improves Function of Exhausted CAR T Cells In Vivo

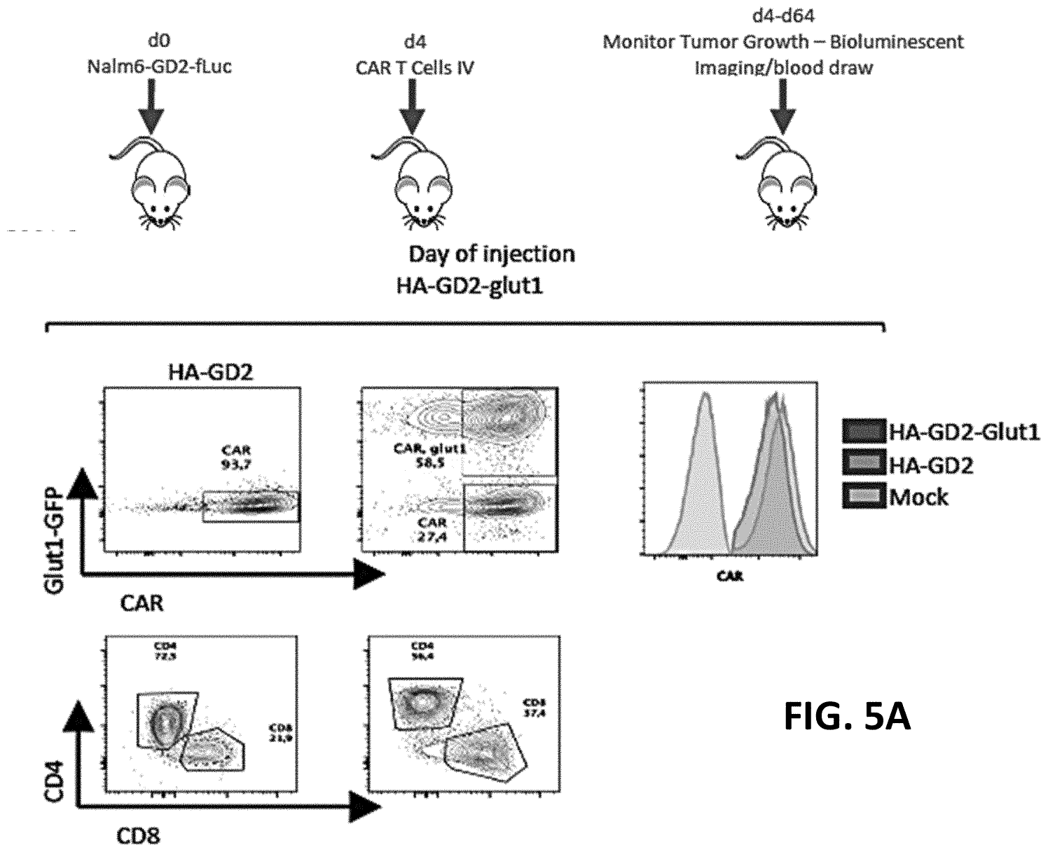

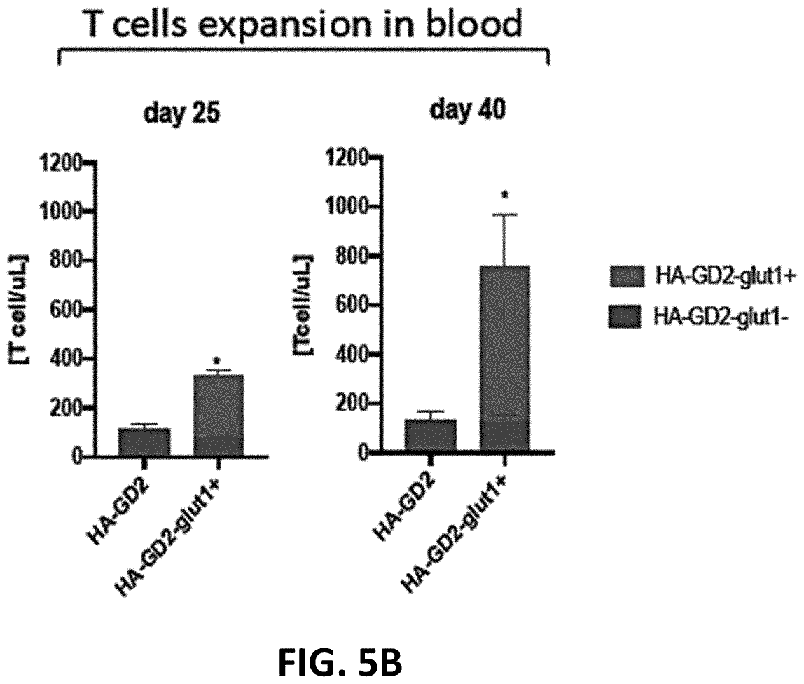

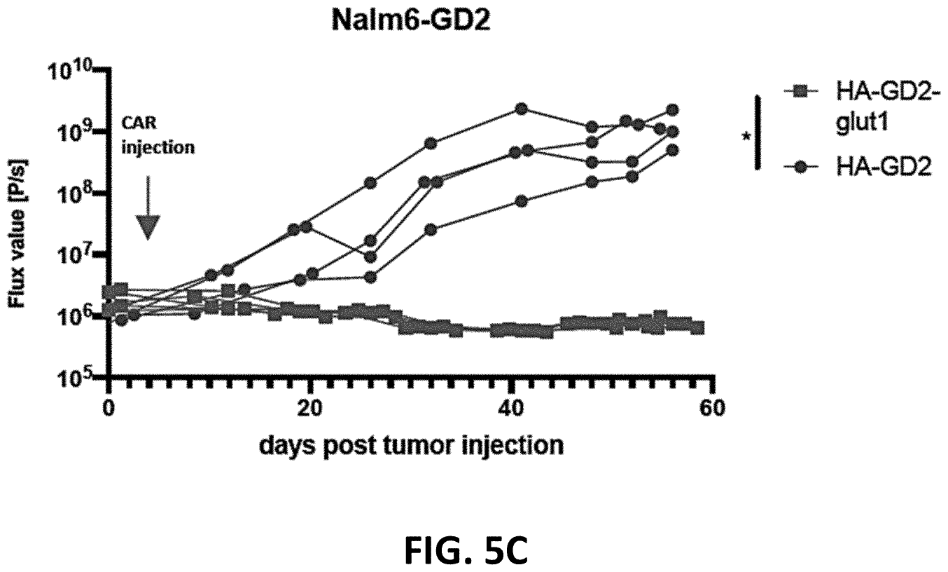

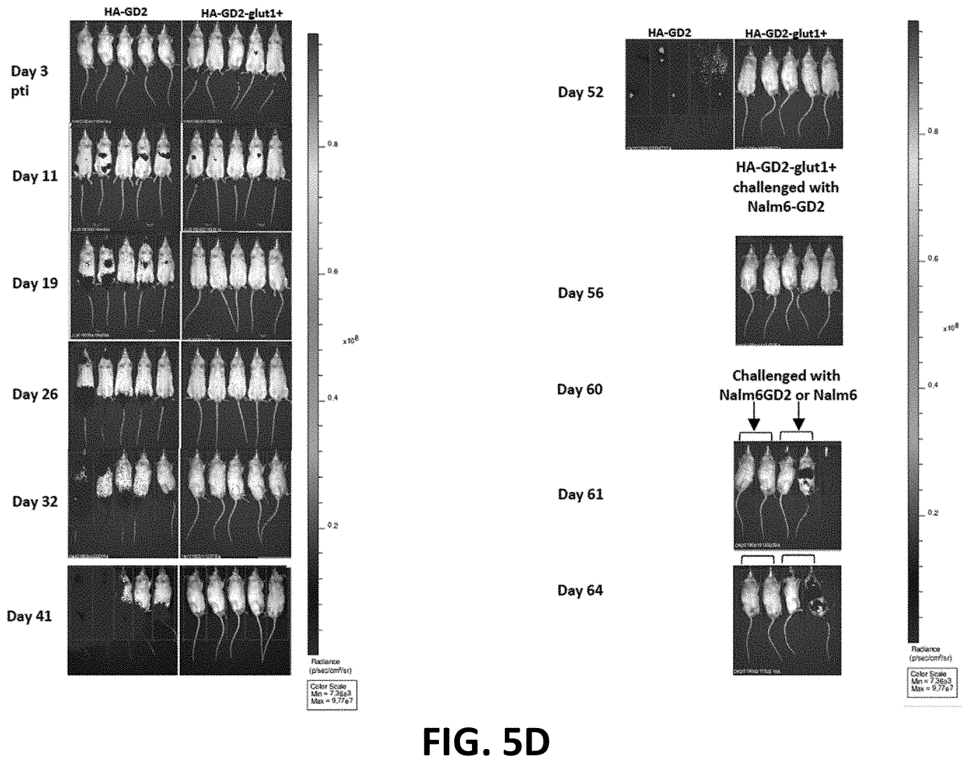

This Example describes the results of experiments performed to illustrate that co-expression of GLUT1 improves function of exhausted CAR T cells in vivo. In these experiments, immunocompromised Nod/SCID/IL2Rg⁻⁻ (NSG) mice were inoculated with 1×10⁶ Nalm6-GD2+ firefly luciferase (fLuc) tumor cells IV on Day 0. Four days post tumor injection, mice were evaluated and normalized for engraftment using bioluminescent imaging. Five mice per group were injected IV with 3×10⁶ GD2 CAR T cells. 3×10⁶ GD2 co-transduced with GLUT1 CART cells on Day 4. At the time of injection, 58% of CAR+ T cells were also GFP-GLUT1⁺ (see, e.g., FIG. 5A). Blood sample analysis at Days 25 and 40 demonstrated enhanced expansion and persistence of GLUT1+GD2 CART cells as compared to HA only CART cells (see, e.g., FIG. 5B). Tumor growth evaluation using weekly bioluminescent imaging showed that mice injected with GLUT1+GD2 CAR T cells exhibited complete tumor clearance by Day 19 post-tumor injection (pti), whereas mice receiving GD2 CAR T cells showed no response to the treatment (see, e.g., FIG. 5C, left panel).

To test the persistence of the GLUT1+GD2 CAR T cells, the mice with Nalm6-GD2+ leukemia were re-challenged at the Day 52. 4 days post re-challenge (i.e., Day 64), no signal of tumor could be detected (see, e.g., FIG. 5C, right top panel) indicating that GLUT1+GD2 CAR T cells were still able to protect against tumors expressing same antigen at the late time point. To assess if this protection was antigen-specific, the mice again were re-challenged again (Day 60) with Nalm6 or Nalm6-GD2+tumor line. Four days later, bioluminescence imaging revealed that only mice challenged with Nalm6 cells that did not express the antigen engrafted, while the GLUT1+GD2 CAR T cells were still persisting and active against Nalm6-GD2+ tumor cells (see, e.g., FIG. 5C, right bottom panels).

While particular alternatives of the present disclosure have been disclosed, it is to be understood that various modifications and combinations are possible and are contemplated within the true spirit and scope of the appended claims. There is no intention, therefore, of limitations to the exact abstract and disclosure herein presented.

### Example 6

### GLUT1 Overexpression Causes Changes at Transcriptome Level in Exhausted CAR T Cells

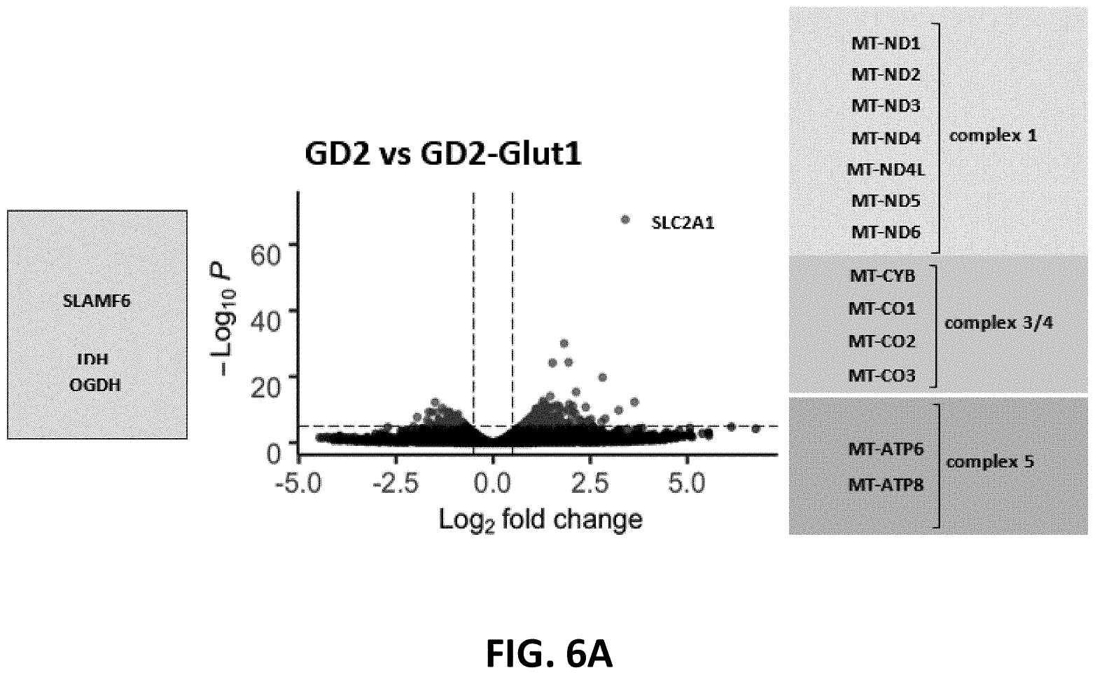

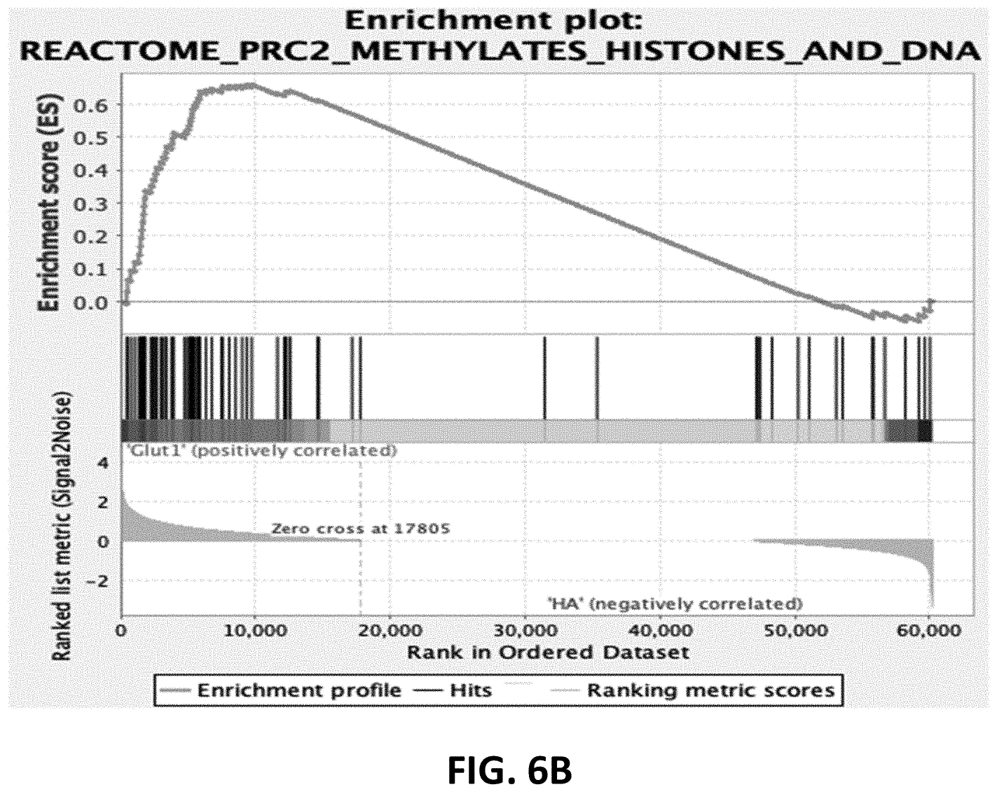

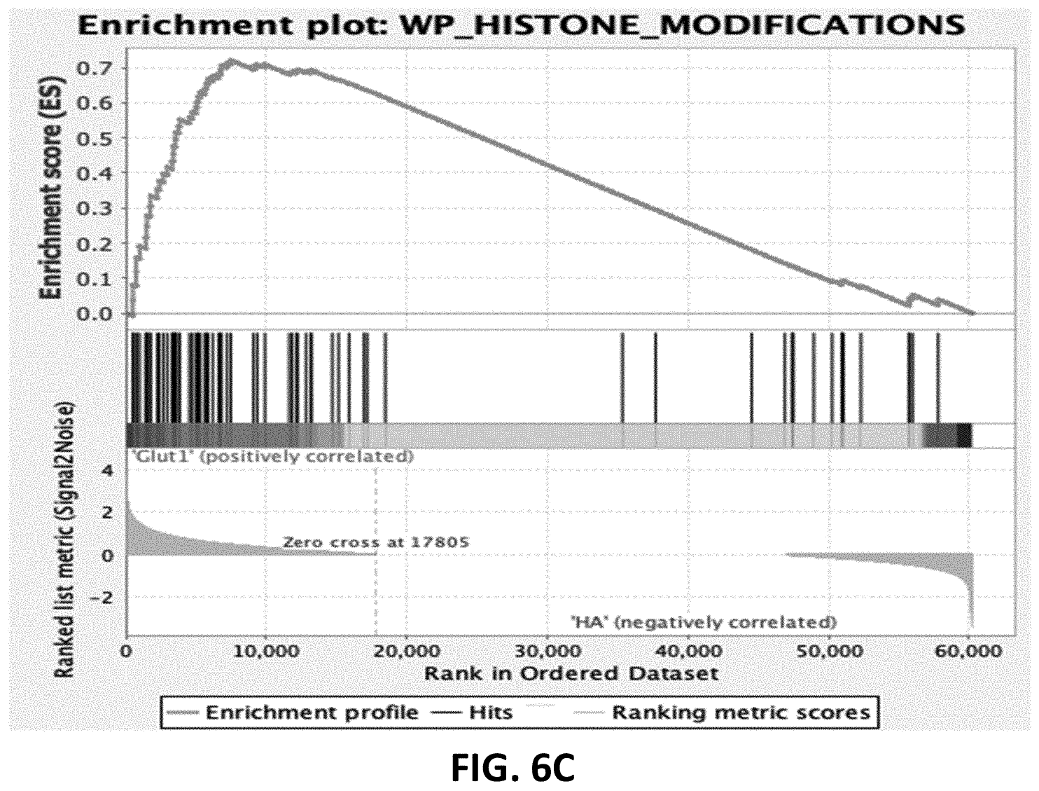

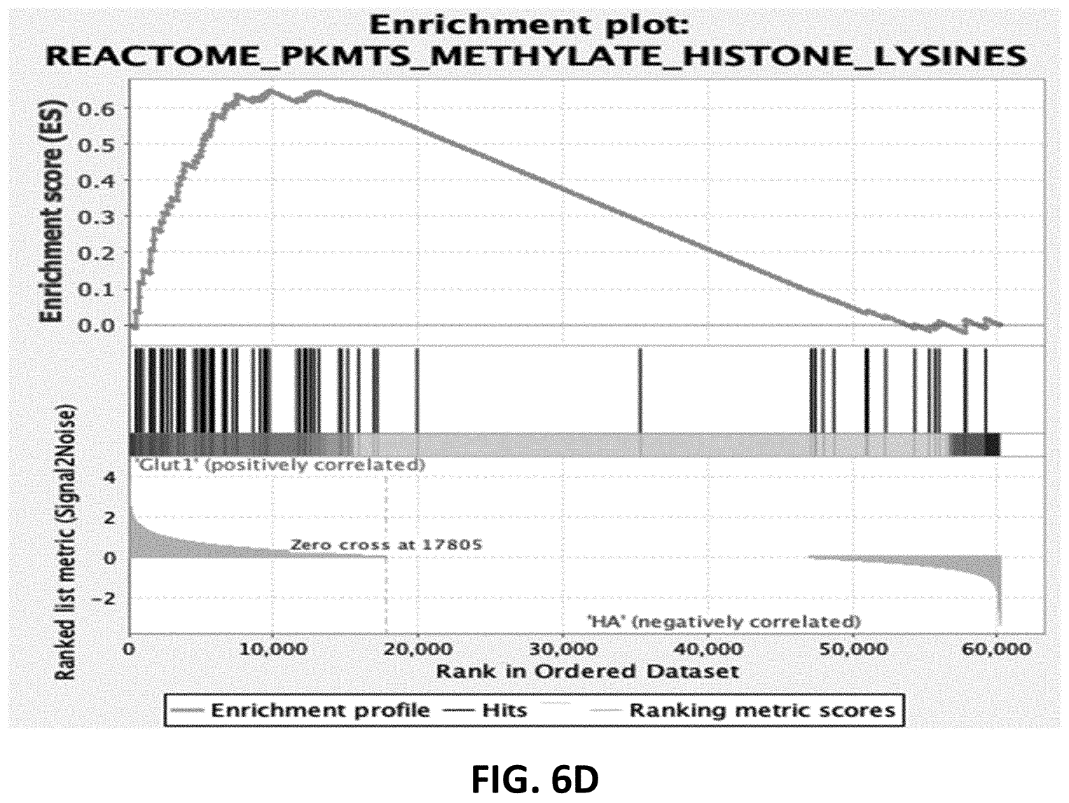

This Example describes the results of experiments performed to illustrate that GLUT1 overexpression causes changes at transcriptome level in exhausted CAR T cells. Transcriptomic profiling of HA-GD2-GLUT1 CAR T cells showed that GLUT1 overexpression increased expression levels of genes involved in electron transport chain in mitochondria, which indicates high mitochondrial activity (see, e.g., FIG. 6A). Remarkably, among the most downregulated genes in GLUT1 overexpressing HA cells was Slam6, a signaling lymphocyte activation molecule family 6 that has been characterized recently as one of the exhaustion regulators. Isocitrate dehydrogenase 1 (IDH1) was another gene that was significantly decreased in HA-GD2-GLUT1 T cells. Given that the role of IDH1 in regulating α-ketoglutarate as a well-known epigenetic regulator, downregulation of IDH1 suggests changes at the epigenetic level. Further, to identify the subset of genes that had the greatest contribution to the enrichment score, a leading-edge analysis on the GSEA datasets was performed. As shown in FIGS. 6B-6D, there was a significant upregulation of genes involved in histone modification, suggesting that GLUT1 overexpression induced epigenetic remodeling.

### Example 7

### GLUT1 Overexpression Increases Mitochondrial Activity

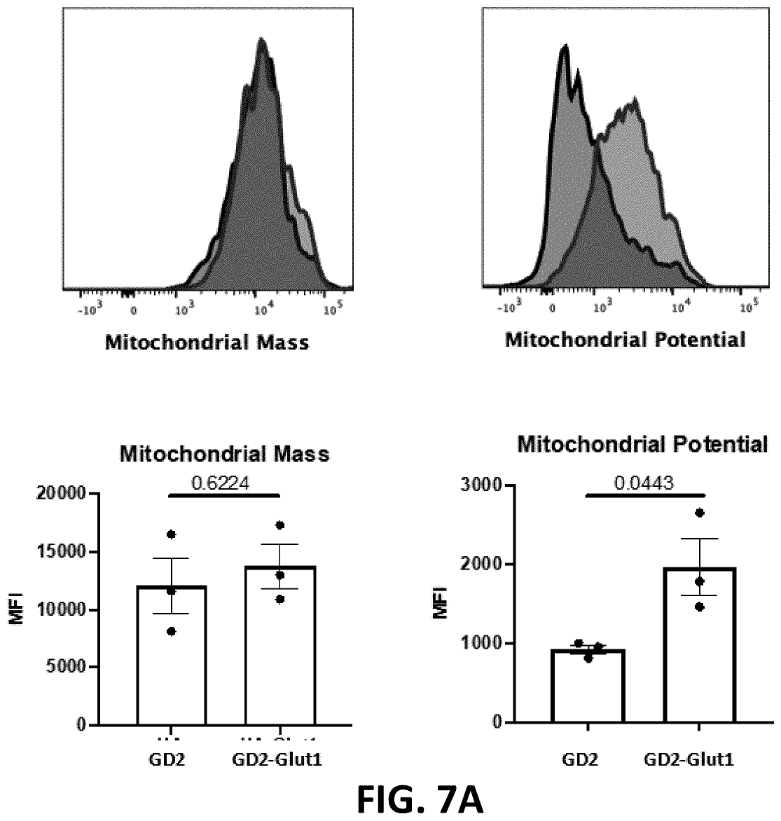

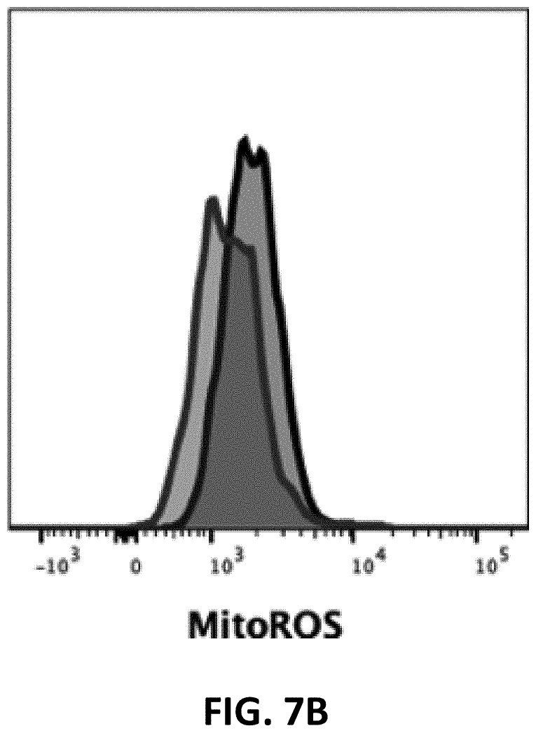

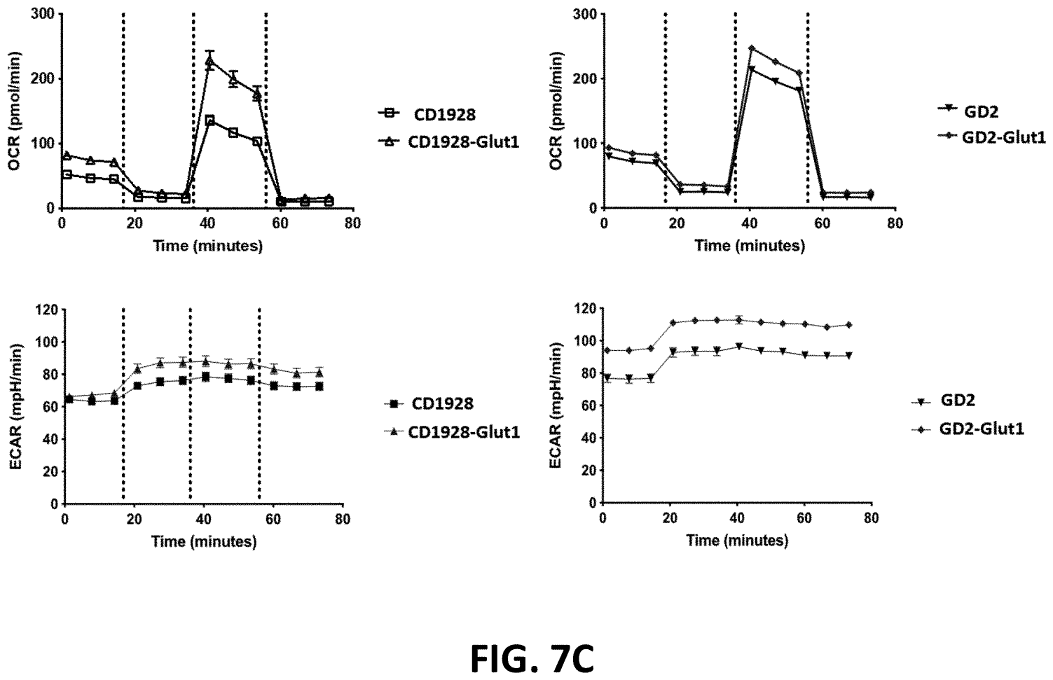

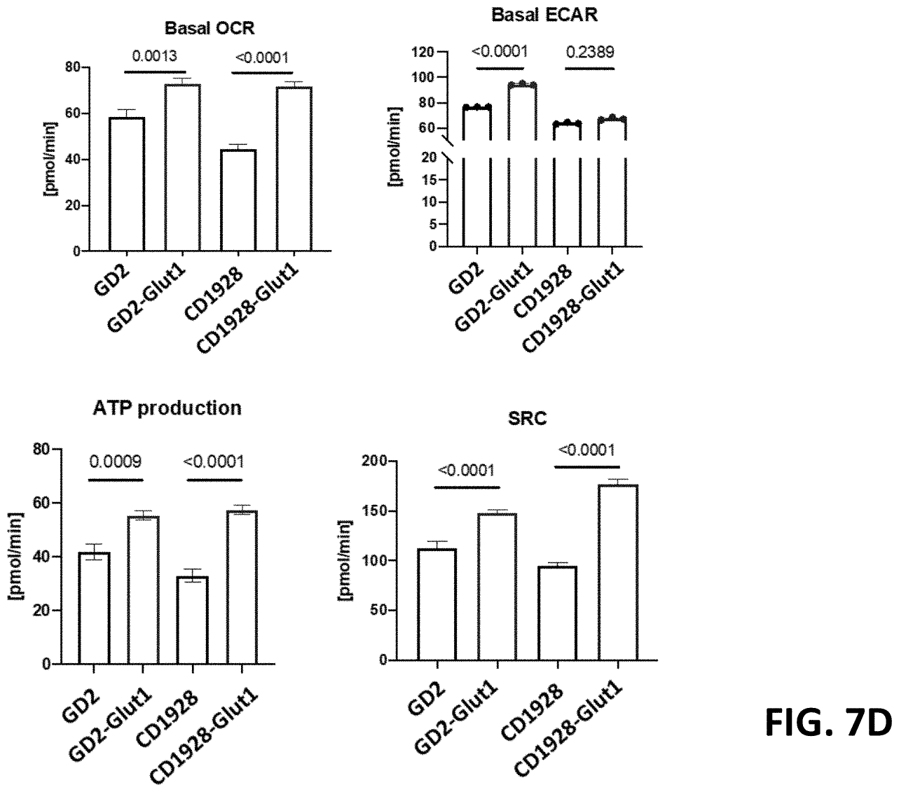

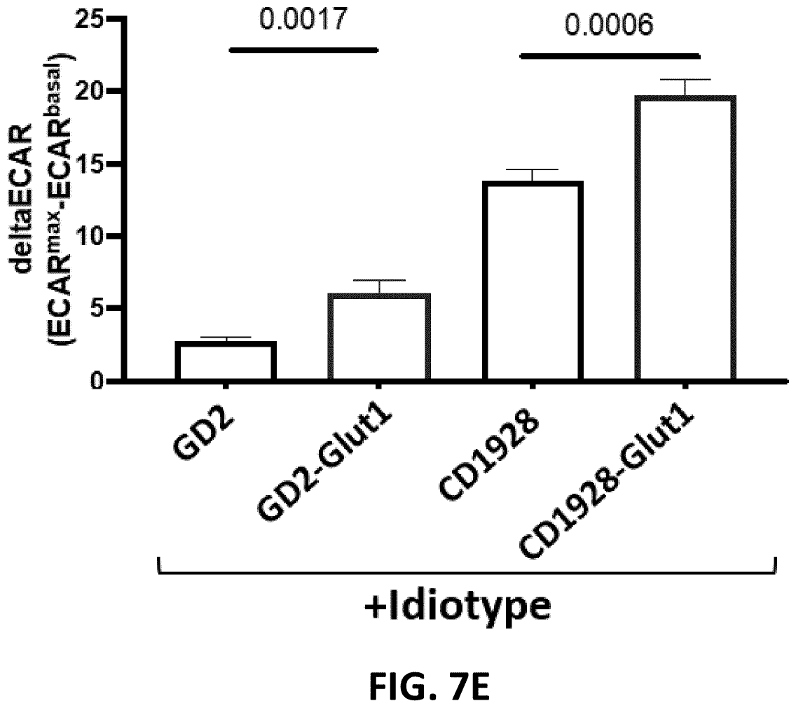

This Example describes the results of experiments performed to illustrate that Glut1 overexpression increases mitochondrial activity. These experiments were designed to investigate how exactly changes in ETC-related genes expression affects mitochondrial function. HA-GD2 and HA-GD2-GLUT1 T cells were stained with MitoTracker Green and MitoTracker Deep Red. The first dye was used as a surrogate for mitochondrial mass and the latter for membrane potential (see, e.g., FIG. 7A). Flow analysis showed that exhausted CAR T cells with overexpressed GLUT1 increased membrane potential, but difference in mitochondrial mass was not detected. This result suggests changes in quality rather than quantity of mitochondria. ROS is a byproduct of normal mitochondrial metabolism and homeostasis; however, buildup of ROS can be potentially damaging by increasing mitochondrial permeability and DNA damage. In addition, with mitoSOX Red dye, it was observed that GLUT1 overexpression decreased the levels of ROS in mitochondria (see, e.g., FIG. 7B).

To further understand how GLUT1 affects mitochondrial function, Mito Stress Test using Seahorse analyzer on exhausted and non-exhausted CAR T cells overexpressing GLUT1 was performed (see, e.g., FIG. 7C). Surprisingly, higher levels of GLUT1 increased basal glycolysis in GD2-HA CAR T cells but not in CD1928 cells. On the other hand, increased basal mitochondrial respiration and ATP linked respiration for both types of CAR T cells were observed, indicating higher mitochondrial activity at the base line. Moreover, Spare Respiratory Capacity (SRC) was increased as well in GLUT1 overexpressing CAR T cells, giving them an extra capacity to produce energy in response to increased stress and as such advantage in the context of cellular survival (FIG. 7D).

The rapid activation-induced glycolysis has been directly linked with T cell effector function. Thus, whether higher levels of GLUT1 is correlated with increased maximal levels of glycolysis upon activation was investigated. For this purpose, CD1928 and HA-GD2 CAR T cells were activated with crosslinked idiotype and ECAR was monitored (see, e.g., FIG. 7E). Although, all CAR T cells engaged glycolysis within minutes of CAR activation, cells overexpressing Glut1 showed enhancement of rapid activation-induced glycolysis (see, e.g., FIG. 7E). This result suggests that GLUT1 overexpressing CAR T cells are able to engage effector response to greater extend after antigen encounter.

### Example 8

### GLUT1 Overexpressing Non-Exhausted CAR T Cells Exhibit Increased Effector Function In Vivo

This Example describes the results of experiments performed to illustrate that GLUT1 overexpressing non-exhausted CAR T cells exhibit increased effector function in vivo. To test the ability of GLUT1 overexpression to improve non-exhausted CAR T cell effector function in vivo, two tumor models were utilized.

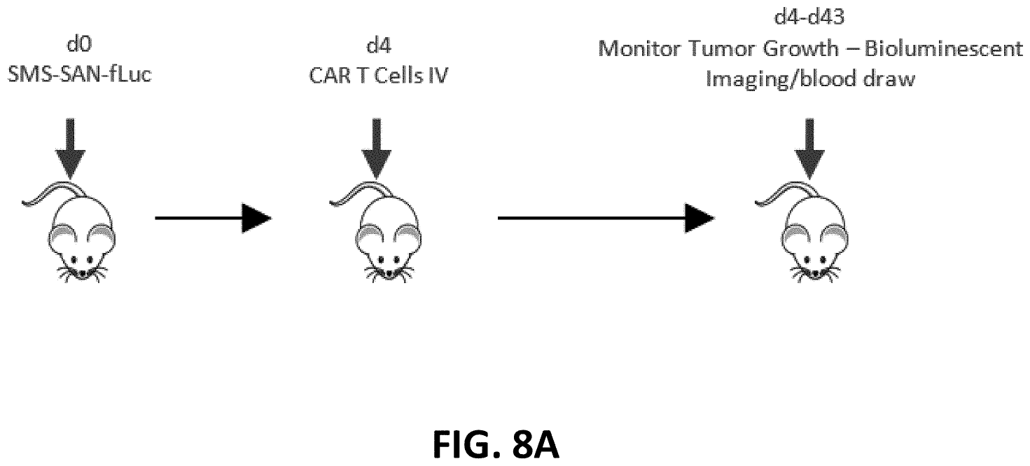

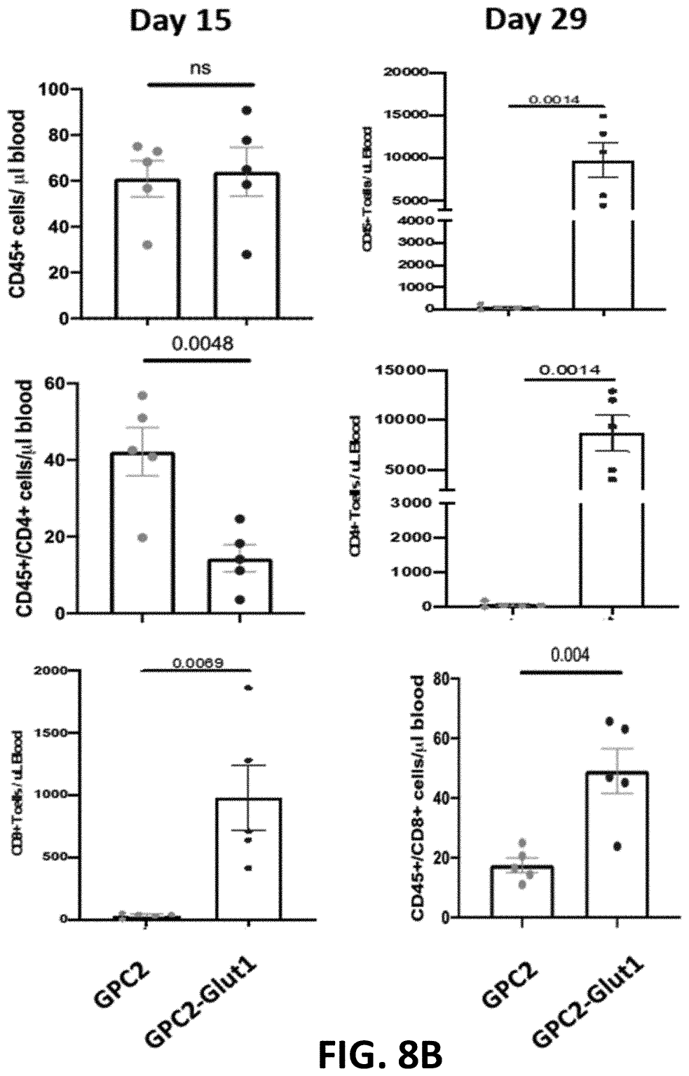

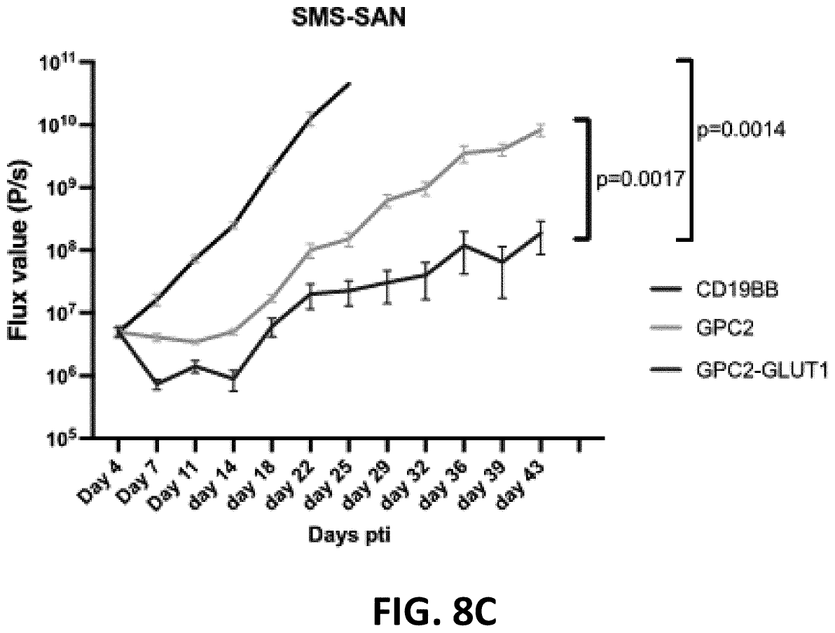

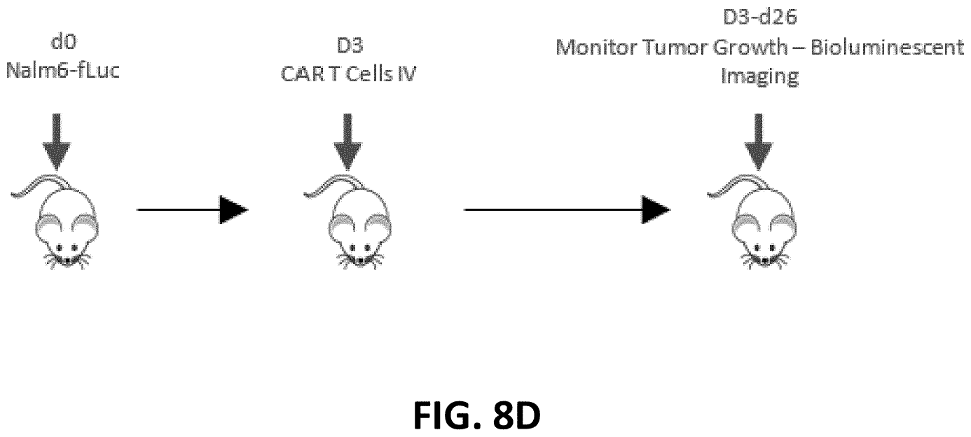

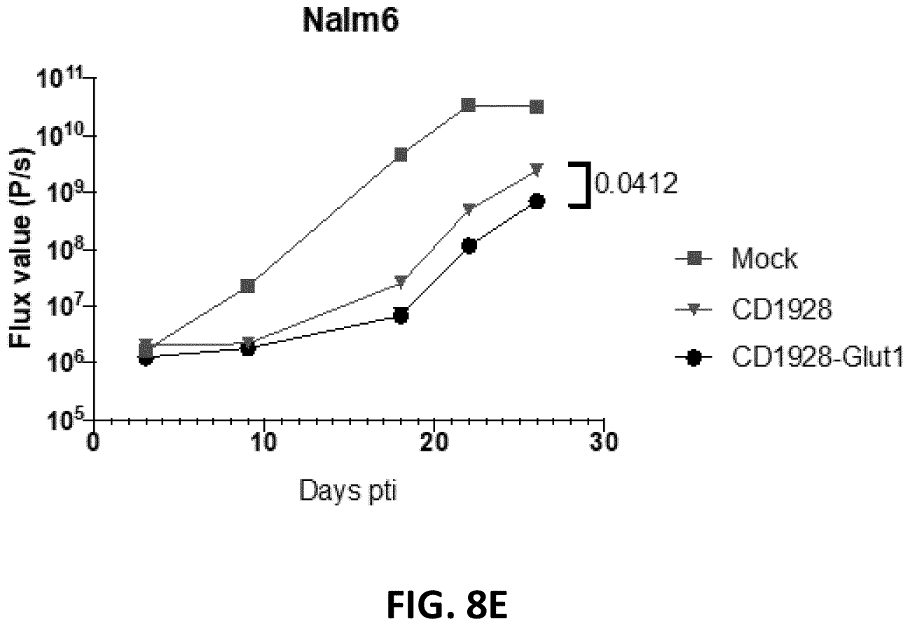

In the first model (FIG. 8A), NSG mice were inoculated with injected IV with 1×10⁶ of SMS-SAN-firefly luciferase (fLuc) neuroblastoma tumor cells. Four days later, mice were injected with 10×10⁶ of CD19bb (negative control), GPC2 or GPC2-Glut1 overexpressing CAR T cells. Although analyzing blood samples at Day 15 post tumor injection showed no significant difference in T cell concentration between the groups, mice injected with GPC2-GLUT1 exhibited higher number of CD8+ T cells. By Day 29, mice injected with GLUT1 overexpressing CAR T cells had significant more T cells than control group (see, e.g., FIG. 8B). This differences in T cell concentration in mice blood correlated with significantly delayed tumor growth in GPC2-GLUT1 mice group (see, e.g., FIG. 8C).

In the second model (FIG. 8D), NSG mice were inoculated with 1×10⁶ Nalm6-firefly luciferase (fLuc) tumor cells IV on Day 0. Four days post tumor injection, mice were evaluated and normalized for engraftment using bioluminescent imaging. Five mice per group were injected IV with 1×10⁶ CD1928 CAR, CD1928-Glut1 CAR T cells on Day 4. Weekly tumor bioluminescent imaging showed that GLUT1 overexpressing CAR T cells significantly delayed tumor growth (see, e.g., FIG. 8E).

### Example 9

### GLUT3 Overexpression Increases Mitochondrial Fitness and Effector Function In Vitro and In Vivo

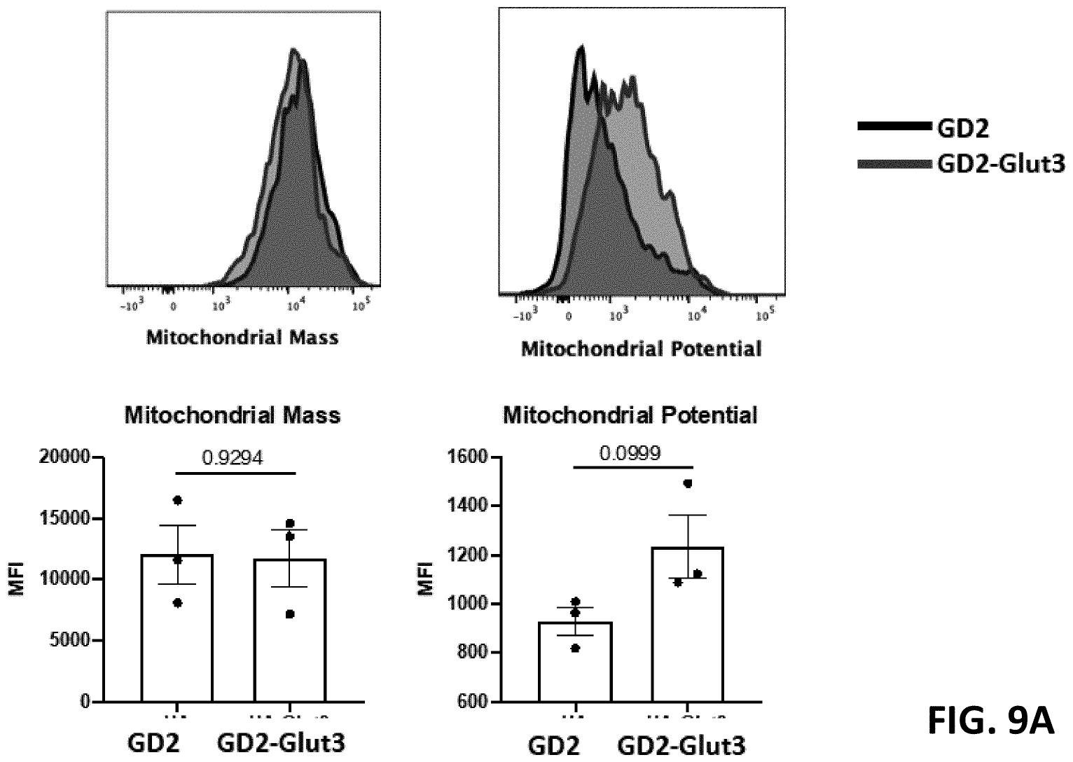

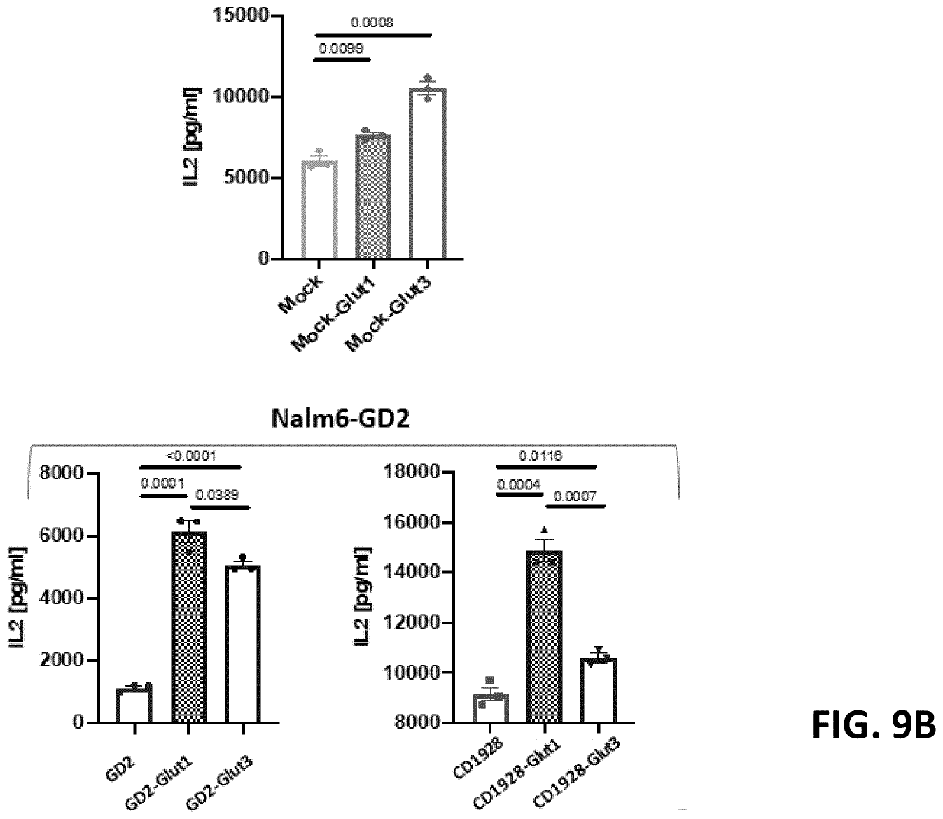

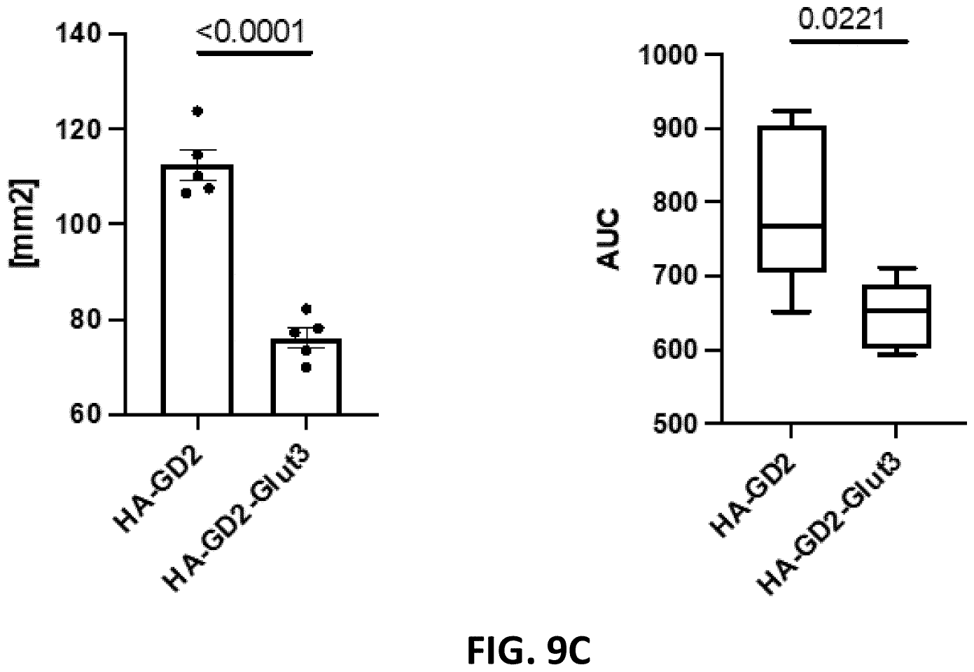

This Example describes the results of experiments performed to illustrate that GLUT3 overexpression increases mitochondrial fitness and effector function in vitro and in vivo. To test if the improvement in CAR T cell function is exclusive to GLUT1 increased expression, additional experiments were performed to overexpress another Glut family glucose transporter, GLUT3 (SLC2a3). In these experiments, it was observed that GLUT3 overexpression restored mitochondrial membrane potential of HA-GD2 exhausted CAR T cells without changing mitochondrial mass (see, e.g., FIG. 9A). Moreover, GLUT3 overexpression augments IL-2 secretion after activation of T cells (mock) upon activation with anti-CD3/CD28 coated beads (1:3 T:B ratio), and both exhausted and non-exhausted CART cells, HA-GD2 and CD1928, respectively, when stimulated with CAR-specific antigen (FIG. 9B). Furthermore, to test GLUT3's capability to improve CAR T cell effector function in vivo, NSG mice were injected with 1×10⁶MG63.3 osteosarcoma tumor cells via intramuscular injections. At Day 24 post injection, 1×10⁶ mock, HA-GD2 or HA-GD2-GLUT3 CAR T cells were injected I.V. It was observed that mice injected with HA-GD2-GLUT3 CAR T cells showed significantly lower tumor burden at Day 47 post tumor injection, and overall faster tumor clearance was observed, depicted as lower area under the curve (AUC) (FIG. 9C). Thus, similar to GLUT1, GLUT3 overexpression improves T cells and CAR T cells effector function and mitochondrial fitness. Without being bound to any particular theory, this improvement translates into better in vivo tumor killing.

While particular alternatives of the present disclosure have been disclosed, it is to be understood that various modifications and combinations are possible and are contemplated within the true spirit and scope of the appended claims. There is no intention, therefore, of limitations to the exact abstract and disclosure herein presented.

What is claimed is:

1. A method for generating an engineered immune cell with enhanced effector function, the method comprising introducing into the immune cell a nucleic acid and/or a polypeptide capable of conferring increased expression of one or more glucose transporters in the immune cell.

2. The method of claim 1, wherein the nucleic acid comprises a sequence encoding one or more glucose transporters.

3. The method of any one of claims 1-2, wherein the one or more glucose transporters is selected from the group consisting of GLUT1, GLUT2, GLUT3, GLUT4, GLUT6, GLUT8, GLUT9, GLUT10, GLUT11, GLUT12, and functional variants of any thereof.

4. The method of any one of claims 1-3, wherein the one or more glucose transporters is selected from the group consisting of GLUT1, GLUT2, GLUT3, GLUT4, and functional variants of any thereof.

5. The method of any one of claims 1-4, wherein the coding sequence of the one or more glucose transporters is operably linked to a transcription control element.

6. The method of any one of claims 1-5, wherein the nucleic acid is incorporated into an expression cassette or a vector.

7. The method of claim 6, wherein the vector is a viral vector derived from a lentivirus, an adeno-virus, an adeno-associated virus, a baculovirus, or a retrovirus.

8. The method of claim 6, wherein the nucleic acid is incorporated into a nucleic construct for use in CRISPR-mediated knock-in procedure.

9. The method of any one of claims 1-8, wherein the immune cell is T lymphocyte, a natural killer (NK) cell, or a natural killer T cell (NKT).

10. The method of claim 9, wherein the T lymphocyte is a CD8+ T cytotoxic lymphocyte cell selected from the group consisting of naïve CD8+ T cells, central memory CD8+ T cells, effector memory CD8+ T cells, effector CD8+ T cells, CD8+ stem memory T cells, bulk CD8+ T cells.

11. The method of claim 9, wherein the lymphocyte is a CD4+ T helper lymphocyte cell selected from the group consisting of naïve CD4+ T cells, central memory CD4+ T cells, effector memory CD4+ T cells, effector CD4+ T cells, CD4+ stem memory T cells, and bulk CD4+ T cells.

12. The method of any one of claims 1-11, wherein the enhanced effector function is selected from the group consisting of growth rate (proliferation), death rate, death rate type, target cell inhibition (cytotoxicity), target cell killing, target cell survival, cluster of differentiation change, macrophage activation, B cell activation, cytokine production, in vivo persistence, and increased glycolytic flux.

13. The method of any one of claims 1-12, wherein the enhanced effector function comprises increased production of interferon gamma (INFγ), tumor-necrosis factor α (TNFα), and/or interleukin-2 (IL-2).

14. The method of any one of claims 1-13, wherein the increased expression of one or more glucose transporters in the immune cell confers increased mitochondrial activity and/or increased mitochondrial fitness of the immune cell.

15. The method of any one of claims 1-14, further comprising introducing into the immune cell one or more recombinant immune receptors.

16. The method of claim 15, wherein the one or more recombinant immune receptors comprises a chimeric antigen receptor (CAR) and/or a T cell receptor (TCR).

17. An engineered immune cell produced by a method according to any one of claims 1-16.

18. The engineered cell of claim 17, wherein the immune cell is in vitro, ex vivo, or in vivo.

19. The engineered cell of any one of claims 17-18, wherein the immune cell is an exhausted immune cell or a non-exhausted immune cell.

20. A cell culture comprising at least one engineered cell of any one of claims 17-19, and a culture medium.

21. A pharmaceutical composition comprising a pharmaceutically acceptable excipient and
a) an engineered cell according to any one of claims 17-19; and/or
b) a nucleic acid comprising a sequence encoding one or more glucose transporters selected from the group consisting of GLUT1, GLUT2, GLUT3, GLUT4, GLUT6, GLUT8, GLUT9, GLUT10, GLUT11, GLUT12, and functional variants of any thereof.

22. The pharmaceutical composition of claim 21, wherein the composition comprises an engineered cell according to any one of claims 17-19, and a pharmaceutically acceptable excipient.

23. The pharmaceutical composition of claim 21, wherein the composition comprises a nucleic acid encoding one or more glucose transporters, and a pharmaceutically acceptable excipient.

24. The pharmaceutical composition of claim 23, wherein the composition comprises the nucleic acid is encapsulated in a viral capsid, a liposome, or a lipid nanoparticle (LNP).

25. A method for treating a health condition in a subject in need thereof, the method comprising administering to the subject a composition comprising:
a) an engineered immune cell according to anyone of claims 17-19;
b) a nucleic acid comprising a sequence encoding one or more glucose transporters selected from the group consisting of GLUT1, GLUT2, GLUT3, GLUT4, GLUT6, GLUT8, GLUT9, GLUT10, GLUT11, GLUT12, and functional variants of any thereof; and/or
c) a pharmaceutical composition according to any one of claims 21-24.

26. The method of claim 25, wherein the health condition is a proliferative disease, an autoimmune disease, or an infection.

27. The method of any one of claims 25-26, wherein the subject is a mammalian subject.

28. The method of any one of claims 25-27, wherein the subject has or is suspected of having a proliferative disease, an autoimmune disease, or an infection.

29. The method of claim 28, wherein the proliferative disease is a cancer.

30. The method of any one of claims 25-29, wherein the administered composition confers enhanced effector function selected from the group consisting of growth rate (proliferation), death rate, death rate type, target cell inhibition (cytotoxicity), target cell killing, target cell survival, cluster of differentiation change, macrophage activation, B cell activation, cytokine production, in vivo persistence, and increased glycolytic flux.

31. The method of claim 30, wherein the enhanced effector function comprises increased production of interferon gamma (INFγ), tumor-necrosis factor α (TNFα), and/or interleukin-2 (IL-2).

32. The method of any one of claims 22-31, wherein the composition is administered to the subject individually (monotherapy) or in combination with a second therapy, wherein the second therapy is selected from the group consisting of chemotherapy, radiotherapy, immunotherapy, hormonal therapy, toxin therapy, or surgery.

33. A kit for the prevention and/or treatment of a condition in a subject in need thereof, the kit comprising:
a) an engineered immune cell according to anyone of claims 17-19;
b) a nucleic acid comprising a sequence encoding one or more glucose transporters selected from the group consisting of GLUT1, GLUT2, GLUT3, GLUT4, GLUT6, GLUT8, GLUT9, GLUT10, GLUT11,GLUT12, and functional variants of any thereof; and/or
c) a pharmaceutical composition according to any one of claims 21-24.
